(19) United States
(12) Patent Application Publication
(10) Pub. No.: US 2024/0261462 A1
(43) Pub. Date: Aug. 8, 2024

(54) SYSTEM AND METHODS FOR USING TISSUE-ADHESIVE POROUS HEMOSTATIC PRODUCTS WITH SEVERE SURFACE AND CAVITY BLEEDING

(71) Applicants: Ethicon, Inc., Somerville, NJ (US); Cilag GmbH International, Zug (CH)

(72) Inventors: Stuart HEAD, Zug (CH); Johannes Caspar Mathias Elizabeth BENDER, Nijmegen (NL); Edwin Alexander ROOZEN, Zug (CH); Erez ILAN, Zug (CH); Rosa Pilar FÉLIX LANAO, Zug (CH); Abraham KEEREWEER, Somerville, NJ (US); Gerard LLANOS, Somerville, NJ (US); Joost OPSTEEN, Zug (CH)

(21) Appl. No.: 18/431,485

(22) Filed: Feb. 2, 2024

Related U.S. Application Data
(60) Provisional application No. 63/483,051, filed on Feb. 3, 2023.

(51) Int. Cl. A61L 24/04

(52) U.S. Cl. CPC A61L 24/046; A61L 2300/802; A61L 2400/04

(57) ABSTRACT

Methods and systems for using a biocompatible and flexible hemostatic sheet comprising a fibrous carrier structure, and reactive electrophilic groups capable of reacting with amine groups in tissue and blood, the hemostatic sheet delivered to the tissue at the bleeding site of an organ to restore hemostasis during an open surgery procedure.

### CROSS-REFERENCE TO RELATED APPLICATIONS

This application claims priority to U.S. Provisional Application Ser. No. 63/483,051, filed Feb. 3, 2023, the disclosure of which is hereby incorporated herein by reference in its entirety for all purposes.

### FIELD

This disclosure relates to devices and methods of applying medical products comprising biocompatible, covalently cross-linked polymers for reduction at bleeding sites.

### BACKGROUND

Intraoperative bleeding can cause significant morbidity and mortality and may have several health economic implication such as longer operative times, more resources' use and longer hospital stay. Hemostatic techniques are essential during surgery or other invasive procedures to provide hemostasis quickly and efficiently. Failure to achieve hemostasis can prolong surgery, impair wound healing, increase infection, and result in unanticipated exposure to blood products if the patient needs a transfusion. When standard methods (e.g., temporary tamponing, electrocautery, and suturing) fail to control bleeding, hemostatic products are used. Topical hemostatic agents such as patches, glues, powders and sprays are divided into three categories: (1) adhesive, containing fibrinogen and thrombin; (2) mechanical, containing gelatin, collagen or oxidative cellulose; and (3) sealants containing polyethylene glycol (PEG). Current products have several disadvantages regarding applicability, user friendliness, and financial costs. Examples of these disadvantages are partial detachment when applied at irregular tissue surfaces, a long waiting time before hemostasis, less effectiveness in coagulopathy conditions and dependency on human-derived coagulation components increasing costs.

While different hemostatic agents are effective in less severe, oozing bleeding from capillaries and small vessels, only a minority of these agents can be applied on active, severe bleedings. Patches are known for their ability to achieve hemostasis in cases of severe bleeding, but their use in cavities or craters is insufficient because they lack flexibility and homogeneity to be shaped to fill a cavity or to conform with highly irregular craters to deliver the coagulating matrix evenly. Flowable hemostatic agents are considered to be the standard-of-care as an adjunct to hemostasis for this type of challenging bleeding, but in severe bleeding cases, flowable agents require additional dosages for complete hemostasis that reduce efficacy and increase time to control of active bleeding.

In view of these clear performance disadvantages, there is a need for flexible, hemostats that can suitably be used to minimize hemorrhage during surgical procedures. The solution of this disclosure resolves these and other issues of the art.

### SUMMARY

The subject of this disclosure is the use of a biocompatible and flexible hemostatic sheet for restoring hemostasis to a tissue at a bleeding site of an organ during a surgical procedure.

An example method of treating hemorrhage in a subject during a surgical procedure. The method can include positioning a hemostatic patch in contact with a tissue at a bleeding site of a respective subject in a first plurality of subjects and restoring hemostasis of the tissue within at least three minutes. The hemostatic patch can include a carrier structure and reactive electrophilic groups capable of reacting with amine groups in tissue and blood.

The present disclosure includes a method for treating hemorrhage in a subject during a surgical procedure. The method can include delivering a first hemostatic patch in contact with a tissue at a bleeding site of an organ of a respective subject in a first plurality of subjects, reacting with the nucleophilic polymer and amine groups in tissue and blood; and restoring hemostasis of the organ within at least three minutes. The first hemostatic patch can include a three-dimensional interconnected interstitial space. The three-dimensional interconnected interstitial space can include a plurality of reactive polymer particles including a nucleophilic polymer carrying reactive nucleophilic groups, and an electrophilic polymer carrying at least three reactive electrophilic groups capable of reacting with amine groups in tissue and blood.

The present disclosure includes a method for treating hemorrhage in a subject during a surgical procedure. The method can include delivering a first hemostatic patch near or about a bleeding site of an organ of a respective subject in a first plurality of subjects and achieving hemostasis of the organ within approximately one minute. The first hemostatic patch can include a nucleophilic polymer carrying reactive nucleophilic groups, and an electrophilic polymer carrying at least three reactive electrophilic groups capable of reacting with the nucleophilic polymer and amine groups in tissue and blood.

The present disclosure includes a device for treating surgical hemorrhage in a subject. The device can include a biocompatible, flexible, hemostatic patch. The hemostatic patch can include a nucleophilic polymer carrying reactive nucleophilic groups, and an electrophilic polymer carrying at least three reactive electrophilic groups capable of reacting with the nucleophilic polymer and amine groups in tissue and blood. The hemostatic device can be configured to be delivered to an organ of a subject and restore hemostasis to the organ within approximately three minutes or less after positioning the hemostatic device in contact with tissue at a bleeding site of the organ.

The present disclosure includes a device for treating surgical hemorrhage in a subject. The device can include a biocompatible, flexible, hemostatic device for treating surgical hemorrhage. The hemostatic device can include a water-resistant cohesive fibrous carrier structure. The fibrous carrier structure can include a three-dimensional interconnected interstitial space having a plurality of reactive polymer particles with an electrophilic polymer, and fibers having a nucleophilic polymer carrying reactive nucleophilic groups. The hemostatic device can be capable of being delivered to an organ of a subject and restoring hemostasis to the organ within approximately three minutes or less by positioning the hemostatic device near or about a bleeding site of the organ.

To the accomplishment of the foregoing and related ends, certain illustrative aspects are described herein in connection with the following description and the appended drawings. These aspects are indicative, however, of but a few of the various ways in which the principles of the claimed subject matter may be employed and the claimed subject matter is intended to include all such aspects and their equivalents. Other advantages and novel features may become apparent from the following detailed description when considered in conjunction with the drawings.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

FIG. 1 shows a patient undergoing an open surgery procedure with an example hemostatic patch positioned at a bleeding site, in accordance with an embodiment of the present disclosure.

FIG. 2A shows a perspective view of an example hemostatic patch, in accordance with an embodiment of the present disclosure.

FIGS. 2B and 2C are structures of example reactive particles embedded in hemostatic patch, in accordance with an embodiment of the present disclosure.

FIG. 3 shows a chart of surface bleeding severity scale (SBSS) scores verbal and visual descriptors and corresponding expected interventions.

FIG. 4 shows a representative overview of an example of a study flow used for this disclosure.

FIG. 5 is a table summarizing demographics for an example study of this disclosure.

FIG. 6A is a table summarizing disease diagnosis for the plurality of subjects in the first study of this disclosure.

FIG. 6B is a table summarizing type of procedure for the plurality of subjects in the first study of this disclosure.

FIG. 7 is a table summarizing treatment details for the plurality of subjects in the first study of this disclosure.

FIG. 8 is a table summarizing characteristics of subjects undergoing use of adjunct hemostatic agents/techniques prior to application of hemostatic patch to achieve hemostatic control for the plurality of subjects in the first study of this disclosure.

FIG. 9 provides a table summarizing hemostasis endpoints for first treated bleeding site per Stage II patient of the first study of this disclosure.

FIG. 10 provides a table summarizing hemostasis endpoints for all treated bleeding sites (e.g., more than one bleeding site per subject) for Stage II subjects and all patients of the first study of this disclosure.

FIG. 11 provides a graphical illustration of time-to-hemostasis after administering hemostatic patch versus Comparative Device 1 for a plurality of subjects in the second study of this disclosure.

FIG. 12 is a graphical depiction of efficacy of adjusted hemostatic patch compared to a blank, Comparative Device 1, and Comparative Device 2 after a liver punch, liver resection, and spleen resection, in accordance with an embodiment of the present disclosure.

FIGS. 13A and 13B show a summary of results of average time to hemostasis and re-bleed of an example of a study used for this disclosure comparing hemostatic patch versus Comparative Device 4 and Comparative Device 5, in accordance with the fourth study of the present disclosure.

FIG. 14 is a table summarizing initial hemostasis between hemostatic patch and Comparative Device 4 and number of applications of each device used in the fifth study of the present disclosure.

FIG. 15 is a flowchart of an example method for treating hemorrhage in a subject during a surgical procedure by delivering hemostatic patch, in accordance with an embodiment of the present disclosure.

FIG. 16 is a flowchart of an example method for treating hemorrhage in a subject during a surgical procedure by delivering hemostatic patch, in accordance with an embodiment of the present disclosure.

FIG. 17 is a flowchart of an example method for treating hemorrhage in a subject during a surgical procedure by delivering hemostatic patch, in accordance with an embodiment of the present disclosure.

FIG. 18 is a flowchart of an example method for treating hemorrhage in a subject during a surgical procedure by delivering hemostatic patch, in accordance with an embodiment of the present disclosure.

### DETAILED DESCRIPTION

Although example embodiments of the disclosed technology are explained in detail herein, it is to be understood that other embodiments are contemplated. Accordingly, it is not intended that the disclosed technology be limited in its scope to the details of construction and arrangement of components set forth in the following description or illustrated in the drawings. The disclosed technology is capable of other embodiments and of being practiced or carried out in various ways.

It must also be noted that, as used in the specification and the appended claims, the singular forms “a,” “an” and “the” include plural referents unless the context clearly dictates otherwise. By “comprising” or “containing” or “including” it is meant that at least the named compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the disclosed technology. Similarly, it is also to be understood that the mention of one or more components in a device or system does not preclude the presence of additional components or intervening components between those components expressly identified.

As discussed herein, vasculature of a “subject” or “patient” may be vasculature of a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to a human (e.g., rat, dog, pig, monkey, or the like). It should be appreciated that the subject may be any applicable human patient, for example.

As discussed herein, “operator” may include a doctor, surgeon, or any other individual or delivery instrumentation associated with delivery of a clot retrieval device to the vasculature of a subject.

As discussed herein, “hemorrhage” can be understood as a release of blood from a broken blood vessel either inside or outside the body. The terms, “hemorrhage”, “bleeding site”, “rupture”, “blood flow”, “bleeding”, and/or the like, can be and are often used interchangeably throughout this disclosure.

The term “hemostatic patch” as used herein, unless indicated otherwise, refers to a sheet having the ability to stop bleeding from damaged tissue. The hemostatic patch of the present invention may achieve hemostasis by turning blood into a gel and/or by forming a seal that closes off the wound site.

The term “tissue-adhesive” as used herein refers to the ability of the hemostatic patch to cling to tissue due to the formation of covalent bonds between the sheet and the tissue. Formation of these covalent bonds typically requires the presence of water.

The term “water-resistant” or “insoluble” as used herein in relation to the fibrous carrier structure means that this structure is not water soluble and does not disintegrate in water to form a colloidal dispersion, at neutral pH conditions (pH 7) and a temperature of 37° C. In some examples, the fibrous carrier structure can absorb as much as about 35 times its own weight of aqueous solutions prior to transitioning to a colloidal dispersion.

The term “interstitial space” as used herein refers to the void (“empty”) space within the fibrous carrier structure. The interstitial space within the fibrous carrier structure allows the introduction of reactive polymer particles into the structure. Also, blood and other bodily fluids can enter the interstitial space, allowing the water-soluble electrophilic polymer within the reactive polymer particles to dissolve.

The concentration of reactive polymer particles having a diameter in the range of 0.5-100 μm is expressed in % by weight of the fibrous carrier structure per se, i.e. without the reactive polymer particles.

The “water-soluble electrophilic polymer carrying reactive electrophilic groups” that is employed in accordance with the present invention carries at least three reactive groups that are capable of reacting with amine groups in tissue and blood under the formation of a covalent bond. This water-soluble electrophilic polymer has a molecular weight of at least 1 kDa and a solubility in distilled water of 20° C. of at least 50 g/L.

The term “water absorption capacity” as used herein is a measure of the capability of the hemostatic patch to absorb water. The water absorption capacity is determined by weighing a sample of the dry sheet (weight=Wd) followed by immersion of the sample into distilled water (37° C.) for 45 minutes. Next, the sample is removed from the water and water clinging to the outside of the substrate is removed, following which the sample is weighed again (weight=Ww). The water absorption capacity=100%×(Ww−Wd)/Wd. The water adsorption capacity is indicative of the porosity of the substrate as well as of its ability to swell in the presence of water.

The term “collagen” as used herein refers the main structural protein in the extracellular space of various connective tissues in animal bodies. Collagen forms a characteristic triple helix of three polypeptide chains. Depending upon the degree of mineralization, collagen tissues may be either rigid (bone) or compliant (tendon) or have a gradient from rigid to compliant (cartilage). Unless indicated otherwise, the term “collagen” also encompasses modified collagens other than gelatin.

The term “gelatin” as used herein refers to a mixture of peptides and proteins produced by partial hydrolysis of collagen extracted from the skin, bones, and connective tissues of animals such as domesticated cattle, chicken, pigs, and fish. During hydrolysis, the natural molecular bonds between individual collagen strands are broken down into a form that rearranges more easily.

The term “polyoxazoline” as used herein refers to a poly(N-acylalkylenimine) or a poly(aroylalkylenimine) and is further referred to as POx. An example of POx is poly(2-ethyl-2-oxazoline). The term “polyoxazoline” also encompasses POx copolymers.

The present disclosure is related to systems, methods and devices for restoring hemostasis to a bleeding site of tissue, and in particular hemorrhage from an organ during open surgery procedures. Certain features, such as a fibrous carrier structure, can be designed to be flexibly positioned within and/or over a bleeding site to stabilize the bleeding. Certain feature of the hemostatic patch of this disclosure can allow the for easier delivery procedures, reduced time to delivery at the bleeding site, and reduce hemostasis.

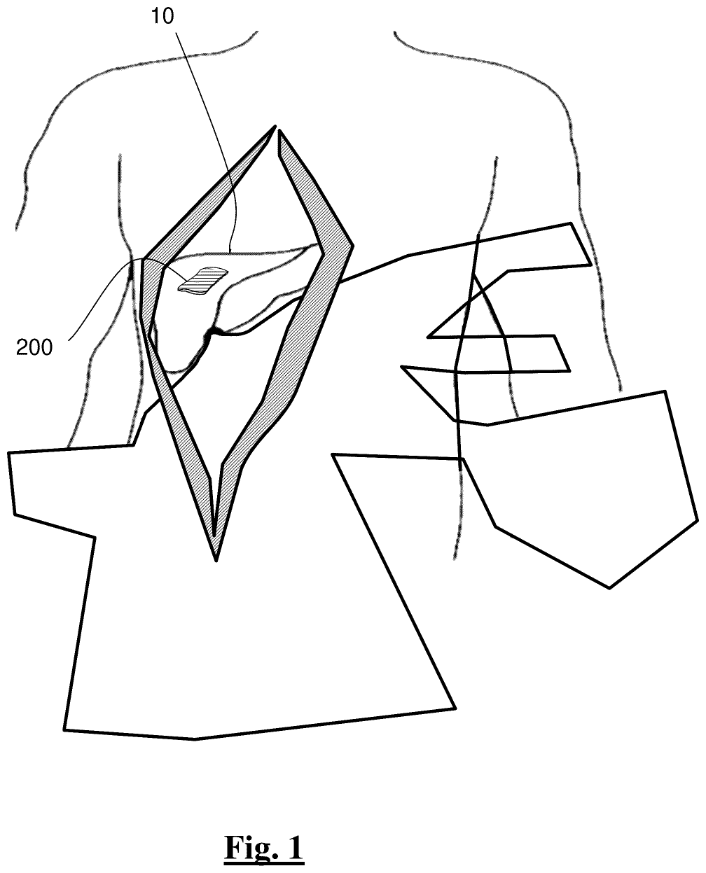

As an example, FIG. 1 depicts a schematic representation of an open surgery procedure with an example hemostatic device 200, also known as a hemostatic patch, positioned at a bleeding site on an organ 10, in particular, the liver. Hemostatic Device 200 can be designed to be positioned within or over any internal bleeding site within the body, such as the liver, pancreas, spleen, stomach, kidney, bladder, reproductive organs, lungs, heart, and the like. Although not shown, Hemostatic Device 200 may also be designed for use during minimally invasive procedures (e.g., laparoscopic) and/or external use (e.g., on the skin of a subject).

Various studies disclosed herein compare device 200 (GATT-Patch 10×5 cm; GATT Technologies BV, Nijmegen, The Netherlands) to those of comparative devices including Comparative Device 1 (FloSeal; Baxter Healthcare, Deerfield, IL, United States); Comparative Device 2 (TachoSil, Corza Medical, Westwood, MA, United States); Comparative Device 3 (Veriset; Medtronic plc, Dublin, Ireland); Comparative Device 4 (Surgifoam; Johnson and Johnson Ethicon, Bridgewater, NJ, United States which is used in the studies described herein in combination with thrombin for more severe bleedings; and Comparative Device 5 (Hemopatch; Baxter International Inc., Deerfield, IL, United States).

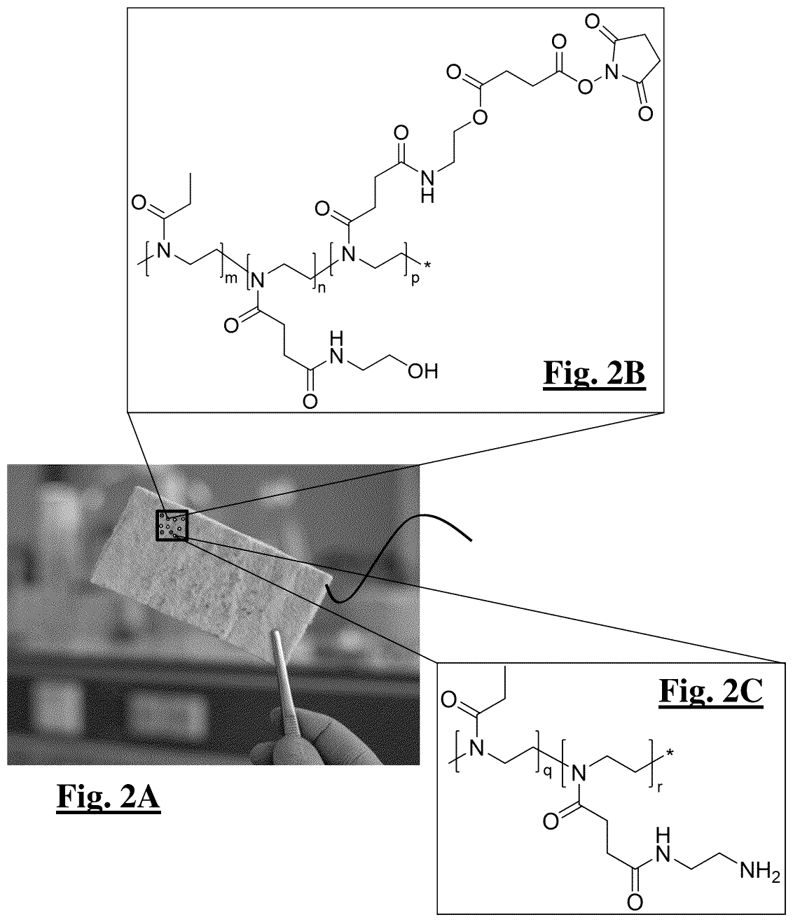

FIG. 2A depicts a pictorial representation of the hemostatic device 200. The Hemostatic Patch 200 according to the present invention can include a water-resistant cohesive fibrous carrier structure that holds small particles. The small particles include a distribution of a reactive electrophilic polymer depicted in FIG. 2B and polymer crosslinks having nucleophiles depicted in FIG. 2C. The reactive electrophilic polymer is capable of covalently binding with host blood proteins and tissue as well as with reactive nucleophilic groups in the polymer crosslinks and in the fibrous carrier structure, and which thereby induces hemostasis and/or tissue-adhesion.

The reactive electrophilic polymer in FIG. 2B includes a functional NHS-ester side chain which can react (i) with amines at the target bleeding site (e.g., tissue, proteins, etc.,), (ii) with the crosslinking polymer of FIG. 2C, and (iii) the amines in the gelatin of the Hemostatic Patch 200 to act as a hemostatic polymer. Crosslinking with tissue results in adhesion to tissue. Crosslinking to gelatin and the crosslinking polymer results in hemostasis within the carrier. The backbone structure (which consists of tertiary amide groups) is generally stable under physiological conditions, but the presence of the ester group renders the side chains intrinsically biodegradable. This ester linkage also contributes to the degradability of the reactive electrophilic polymer. The reactive electrophilic polymer can include a composition of P(EtOx-OH—NHS) and forms a granulate embedded in the Hemostatic Patch 200. The backbone shown in FIG. 2B includes a copolymer having three species of monomer (terpolymer). In some examples, the P(EtOx-OH—NHS) can be formed with a 60-20-20 ratio (of about 60% POx, about 20% OH, and about 20% NHS). Of the 60% Pox, the “Et” component can be inert and limit post polymerisation activation, whereas the “Me” components (40%) can be post polymerisation activated. The first monomer can have chain length m ranging from about 5 to about 5,000 repeating units. The second monomer can have a chain length n ranging from about 5 to about 5,000 repeating units. The third monomer can have a chain length p ranging from about 5 to about 5,000 repeating units. The PDI can range from between about 1.10 to about 2.50.

The crosslinking polymer of FIG. 2C includes amine side changes which can react with the electrophilic polymer of FIG. 2B to act as a crosslinking agent. The backbone structure includes tertiary amide groups and is generally stable under physiological conditions. The crosslinking polymer can include a composition of P(EtOx-NH₂). The backbone shown in FIG. 2C includes a copolymer having two species of monomer (bipolymer). In some examples, the P(EtOx-NH₂) can be formed with a 90-10 ratio (of about 90% POx and about 10% NH₂). The first monomer can have chain length q ranging from about 5 to about 5,000 repeating units. The second monomer can have a chain length r ranging from about 5 to about 5,000 repeating units.

The molecular weight distribution or polydispersity index (PDI) of granulated reactive electrophilic polymer depicted in FIG. 2B and polymer crosslinks depicted in FIG. 2C is below 2.0 at release. Premature or excessive crosslinking between NHS-POx and NU-Pox and/or gelatin results in an increase of the PDI. If this crosslinking occurs, there will be less NHS groups readily available to which can adhere to the tissue and reduce the hemostatic control of Hemostatic Patch 200.

Hemostatic patch 200 can restore hemostasis to a bleeding site by positioning Hemostatic Patch 200 in contact with the tissue at the bleeding site. It is understood that Hemostatic Patch 200 could be used to restore hemostasis to the tissue within three minutes or less (e.g., 30 seconds) after putting Hemostatic Patch 200 in contact with the tissue. As applicable procedure guidelines change with respect to the use of hemostatic patch for treatment of open surgery procedures, it is also conceivable that device 200 could be delivered via alternative techniques, such as during minimally invasive procedures. Hemostatic Patch 200 can be understood as including features that are clearly described in Appendix A from U.S. Pat. Nos. 10,232,077 and 9,416,228, U.S. Publication No. 20220133949 (corresponding to U.S. patent application Ser. No. 17/573,564), US Publication No. 20220133948 (corresponding to U.S. patent application Ser. No. 17/573,541), U.S. Publication No. 20220133947 (corresponding to U.S. patent application Ser. No. 17/573,537); U.S. Publication No. 20220133943 (corresponding to U.S. patent application Ser. No. 17/573,574), and U.S. Publication No. 20220153930 (corresponding to U.S. patent application Ser. No. 17/586,428), each of which are incorporated by reference in their entirety as if set forth verbatim herein.

The Hemostatic Patch 200 includes a water-resistant cohesive fibrous carrier structure that readily absorbs blood as blood can penetrate the interstitial space. This fibrous carrier structure can easily be impregnated with reactive polymer particles. Unlike impregnation with liquids, such dry impregnation does not affect the structural integrity or mechanical properties of the carrier structure. When blood is absorbed by the Hemostatic Patch 200, the reactive polymer particles within the sheet start dissolving as soon as they are ‘wetted’ by the blood, thereby allowing the electrophilic polymer to react with both reactive nucleophilic groups in the blood and tissue and reactive nucleophilic groups in the fibrous carrier structure, thereby inducing blood coagulation and tissue sealing, both of which contribute to hemostasis.

The reactive polymer particles may be homogeneously distributed within the interstitial space of the fibrous carrier structure in the sense that the particle density is essentially the same throughout the carrier structure. Alternatively, the reactive polymer particles may also be unevenly distributed throughout the carrier structure. For instance, if the hemostatic sheet is prepared in the form of a laminate of thin layers of fibrous carrier structure and layers of reactive polymer particles, the reactive polymer particle density within the sheet may fluctuate. For certain applications it may be advantageous if the reactive polymer particle density shows a gradient, e.g. in that the density of reactive particles is highest near the side of the sheet that is meant to applied onto a bleeding wound and lowest near the other side of the sheet.

The diameter distribution of the reactive polymer particles may suitably be determined by means of laser diffraction using a Malvern Mastersizer 2000 in combination with the Stainless-Steel Sample Dispersion Unit. The sample dispersion unit is filled with approx. 120 ml of cyclohexane, which is stabilized for 5 to 10 minutes at a stirring speed of 1800 rpm, followed by a background measurement (blank measurement). The sample tube is shaken and turned horizontally for 20 times. Next, about 50 mg is dispersed in the sample dispersion unit containing the cyclohexane. After the sample is introduced in the dispersion unit, the sample is stirred for one and a half minute at 1800 rpm to ensure that all particles are properly dispersed, before carrying out the measurement. No ultrasonic treatment is performed on the dispersed particles. Mean particle size is expressed as D [4,3], the volume weighted mean diameter (ΣnᵢDᵢ⁴)/(ΣnᵢDᵢ³).

In a particularly preferred embodiment, unlike the fibrous tissue sealant described in U.S. Pat. No. 8,545,871, the hemostatic patch of the present invention does not form a hydrogel, until it is wetted upon delivery to the bleeding site i.e. a water-swellable polymeric matrix that can absorb a substantial amount of water to form an elastic gel.

According to a particularly preferred embodiment, the Hemostatic Patch 200 is bioabsorbable, meaning that the carrier structure, the reactive polymer particles, and any other components of the Hemostatic Patch 200 are eventually absorbed in the body. Absorption of the carrier structure and reactive polymer particles typically requires chemical decomposition (e.g. hydrolysis) of polymers contained therein. Complete bioabsorption of the Hemostatic Patch 200 by the human body is typically achieved in approximately 1 to 10 weeks, preferably in approximately 4 to 6 weeks.

The Hemostatic Patch 200 typically has a non-compressed mean thickness of 0.5-25 mm. More preferably, the non-compressed mean thickness is in the range of 1-10 mm, most preferably in the range of 1.5-5 mm.

The dimensions of the Hemostatic Patch 200 preferably are such that the top and bottom of the sheet each have a surface area of at least 2 cm², more preferably of at least 10 cm²and most preferably of 25-50 cm². Typically, the sheet is rectangular in shape and has a length of 25-200 mm, and a width of 25-200 mm. Due to its flexibility, the Hemostatic Patch 200 of the present invention can suitably be applied to irregularly shaped bleeding sites. The haemostatic sheet may be applied layer on layer if an already applied sheet does not fully stop the bleeding.

Hemostatic patch 200 can be cut into any suitable shape and size for the delivery method for the open surgery procedure or for the size of the bleeding site. In addition, or alternatively thereto, Hemostatic Patch 200 can be shredded or otherwise formed into a construction similar to cotton candy due to the fibrous carrier structure. During a procedure, Hemostatic Patch 200 may be shredded or rolled into a ball and positioned within a cavity at the bleeding site, followed by placement of an unshredded patch over the bleeding site to restore hemostasis.

The Hemostatic Patch 200 preferably has a non-compressed density of less than 200 mg/cm³, more preferably of less than 150 mg/cm³and most preferably of 10-100 mg/cm³.

In one embodiment of the invention the reactive polymer particles are homogeneously distributed within the interstitial space of the fibrous carrier structure. In another embodiment of the invention the Hemostatic Patch 200 is a laminate comprising alternating layers of fibrous carrier structure and layers of the reactive polymer particles. In the latter embodiment, reactive polymer particles preferably have entered the layers of fibrous carrier structure that separate the layers of reactive polymer particles.

The Hemostatic Patch 200 preferably is essentially anhydrous. Typically, the Hemostatic Patch 200 has a water content of not more than 5 wt. %, more preferably of not more than 2 wt. % and most preferably of not more than 1 wt. %. The water absorption capacity of the Hemostatic Patch 200 preferably is at least 50%, more preferably lies in the range of 100% to 800%, most preferably in the range of 200% to 500%.

The Hemostatic Patch 200 of the present invention is preferably sterile.

The reactive polymer particles in the Hemostatic Patch 200 preferably include a water-soluble electrophilic polymer that carries reactive electrophilic groups selected from carboxylic acid esters, sulfonate esters, phosphonate esters, pentafluorophenyl esters, p-nitrophenyl esters, p-nitrothiophenyl esters, acid halide groups, anhydrides, ketones, aldehydes, isocyanato, thioisocyanato, isocyano, epoxides, activated hydroxyl groups, olefins, glycidyl ethers, carboxyl, succinimidyl esters, sulfo succinimidyl esters, maleimido (maleimidyl), ethenesulfonyl, imido esters, aceto acetate, halo acetal, orthopyridyl disulfide, dihydroxy-phenyl derivatives, vinyl, acrylate, acrylamide, iodoacetamide and combinations thereof. More preferably, the reactive electrophilic groups are selected from carboxylic acid esters, sulfonate esters, phosphonate esters, pentafluorophenyl esters, p-nitrophenyl esters, p-nitrothiophenyl esters, acid halide groups, anhyinidrides, ketones, aldehydes, isocyanato, thioisocyanato, isocyano, epoxides, activated hydroxyl groups, glycidyl ethers, carboxyl, succinimidyl esters, sulfo succinimidyl esters, imido esters, dihydroxy-phenyl derivatives, and combinations thereof. Even more preferably, the reactive electrophilic groups are selected from halo acetals, orthopyridyl disulfide, maleimides, vinyl sulfone, dihydroxyphenyl derivatives, vinyl, acrylate, acrylamide, iodoacetamide, succinimidyl esters and combinations thereof. Most preferably, the reactive electrophilic groups are selected from maleimides, vinyl, acrylate, acrylamide, succinimidyl esters, sulfo succinimidyl esters and combinations thereof.

Suitable succinimidyl esters that may be employed include succinimidyl glutarate, succinimidyl propionate, succinimidyl succinamide, succinimidyl carbonate, disuccinimidyl suberate, bis(sulfosuccinimidyl) suberate, dithiobis(succinimidylpropionate), bis(2-succinimidooxycarbonyloxy) ethyl sulfone, 3,3′-dithiobis(sulfosuccinimidyl-propionate), succinimidyl carbamate, sulfosuccinimidyl(4-iodoacetyl)aminobenzoate, bis(sulfosuccinimidyl) suberate, sulfosuccinimidyl-4-(N-maleimidomethyl)-cyclohexane-1-carboxylate, dithiobis-sulfosuccinimidyl propionate, disulfo-succinimidyl tartarate; bis[2-(sulfo-succinimidyloxycarbonyloxyethylsulfone)], ethylene glycol bis(sulfosuccinimiclylsuccinate), dithiobis-(succinimidyl propionate), and combinations thereof.

Suitable dihydroxyphenyl derivatives that may be employed include dihydroxyphenylalanine, 3,4-dihydroxyphenylalanine (DOPA), dopamine, 3,4-dihydroxyhydroccinamic acid (DOHA), norepinephrine, epinephrine and catechol.

The use of a fibrous carrier structure in the Hemostatic Patch 200 offers the advantage that the reactive polymer particles can be homogeneously distributed throughout this carrier structure without difficulty. Such a homogeneous distribution is much more difficult to achieve in, for instance, foamed carrier structures.

The fibers in the fibrous carrier structure preferably have a mean diameter of 1-500 μm, more preferably of 2-300 μm and most preferably of 5-200 μm. The mean diameter of the fibers can suitably be determined using a microscope. Typically, at least 50 wt. %, more preferably at least 80 wt. % of the fibers in the fibrous carrier structure have a diameter of 1-300 μm and a length of at least 1 mm. Preferably, at least 50 wt. %, more preferably at least 80 wt. % of the fibers in the fibrous carrier structure have an aspect ratio (ratio of length to diameter) of at least 1000.

The fibrous carrier structure can include a felt structure, a woven structure, or a knitted structure. Most preferably, the fibrous carrier structure is a felt structure. Here the term “felt structure” refers to a structure that is produced by matting and pressing fibers together to form a cohesive material.

According to an embodiment, the fibrous carrier structure is biodegradable.

The nucleophilic polymer that is contained in the fibrous carrier structure may be homogenously distributed throughout fibers that are contained in the carrier's structure or it maybe applied as an external coating layer. The presence of nucleophilic polymer in the carrier structure improves both adhesion and haemostatic properties of the Hemostatic Patch 200.

Preferably, the fibers of the fibrous carrier structure contain at least 5 wt. %, more preferably at least 10 wt. % and more preferably at least 50 wt. % of the nucleophilic polymer.

Most preferably, the fibers consist of said nucleophilic polymer.

The nucleophilic polymer that is contained in fibers of the carrier structure typically contains at least 2 reactive nucleophilic groups, more preferably at least 5 reactive nucleophilic groups, even more preferably at least 10 reactive nucleophilic groups, most preferably at least 20 reactive nucleophilic groups. These reactive nucleophilic groups are preferably selected from amine groups, thiol groups, phosphine groups and combinations thereof. More preferably, these reactive nucleophilic groups are selected from amine groups, thiol groups and combinations thereof. Most preferably, the reactive nucleophilic groups are amine groups. These amine groups are preferably selected from primary amine groups, secondary amine groups and combinations thereof.

The nucleophilic polymer in the fibers of the fibrous carrier structure preferably has a nitrogen content of at least 1 wt. %, more preferably of 5-10 wt. % and most preferably of 15-25 wt. %.

The nucleophilic polymer is preferably selected from protein, chitosan, synthetic polymer carrying reactive nucleophilic groups, carbohydrate polymers carrying reactive nucleophilic groups and combinations thereof. More preferably, the nucleophilic polymer is selected from gelatin, collagen, chitosan and combinations thereof. Even more preferably, the nucleophilic polymer is gelatin, most preferably cross-linked gelatin.

Chitosan is a biodegradable, nontoxic, complex carbohydrate derivative of chitin (poly-N-acetyl-D-glucosamine), a naturally occurring substance. Chitosan is the deacetylated form of chitin. The chitosan applied in accordance with the present invention preferably has a degree of deacetylation of more than 70%.

The fibrous carrier structure preferably comprises at least 50 wt. %, more preferably at least 80 wt. % and most preferably at least 90 wt. % fibers containing a nucleophilic polymer carrying reactive nucleophilic groups.

In a preferred embodiment, the fibrous carrier structure comprises at least 50 wt. %, more preferably at least 80 wt. % and most preferably at least 90 wt. % fibers containing at least 50 wt. % of a nucleophilic polymer carrying reactive nucleophilic groups. According to a particularly preferred embodiment, the fibrous carrier structure comprises at least 50 wt. %, more preferably at least 80 wt. % and most preferably at least 90 wt. % fibers made from gelatin, collagen, or chitosan.

Preferred collagens do not possess telopeptide regions (“atelopeptide collagen”). The collagen employed in accordance with the present invention is preferably selected from the group of microfibrillar collagen, synthetic human collagen such as the type I collagen, type III collagen, or a combination of type I collagen and type III collagen. Collagen crosslinked using heat, radiation, or chemical agents such as glutaraldehyde may also be used.

In accordance with a preferred embodiment, the fibers in the fibrous carrier structure comprise at least 50 wt. %, more preferably at least 80 wt. % and most preferably at least 90 wt. % gelatin. The gelatin in the fibers preferably has a Bloom strength of 200 or more.

In a particularly advantageous embodiment, the fibrous carrier structure comprises at least 50 wt. %, more preferably at least 80 wt. % and most preferably at least 90 wt. % of partially cross-linked gelatin. The use of partially cross-linked gelatin offers the advantage that the fibrous carrier structure is both sufficiently stable and flexible at body temperature, and that swelling of the fibrous carrier structure does not result in the formation of a closed-pore fibrous gel structure.

In the preparation of the Hemostatic Patch 200, it can be advantageous to react a fraction of the reactive electrophilic groups in the electrophilic polymer of the of the reactive polymer particles with the reactive nucleophilic groups of the nucleophilic polymer. Thus, the reactive polymer particles may be fixated effectively within the fibrous carrier structure.

According to a preferred embodiment, the reactive nucleophilic groups of the nucleophilic polymer in the fibers of the fibrous carrier structure include amine groups and the reactive electrophilic groups of the electrophilic polymer in the reactive polymer particles are selected from carboxylic acid esters, sulfonate esters, phosphonate esters, pentafluorophenyl esters, p-nitrophenyl esters, p-nitrothiophenyl esters, acid halide groups, anhydrides, ketones, aldehydes, isocyanato, thioisocyanato, isocyano, epoxides, activated hydroxyl groups, glycidyl ethers, carboxyl, succinimidyl esters, sulfosuccinimidyl esters,, imido esters, dihydroxy-phenyl derivatives, and combinations thereof.

According to another preferred embodiment, the reactive nucleophilic groups of nucleophilic polymer include thiol groups and the reactive electrophilic groups of the electrophilic polymer in the reactive polymer particles are selected from halo acetals, orthopyridyl disulfide, maleimides, vinyl sulfone, dihydroxyphenyl derivatives, vinyl, acrylate, acrylamide, iodoacetamide, succinimidyl esters, sulfosuccinmidyl esters and combinations thereof. More preferably, the reactive electrophilic groups are selected from succinimidyl esters, sulfosuccinimidyl esters, halo acetals, maleimides, or dihydroxyphenyl derivatives and combinations thereof. Most preferably, the reactive electrophilic groups are selected from maleimides or dihydroxyphenyl derivatives and combinations thereof.

In a preferred embodiment of the invention, the fibrous carrier structure does not comprise oxidised regenerated cellulose.

### Study Overview

This disclosure is more clearly understood with the corresponding studies discussed more particularly below with respect to open surgical procedures of hemorrhaging at different organs. It is understood that data is presented herein for purposes of illustration and should not be construed as limiting the scope of the disclosed technology in any way or excluding any alternative or additional embodiments.

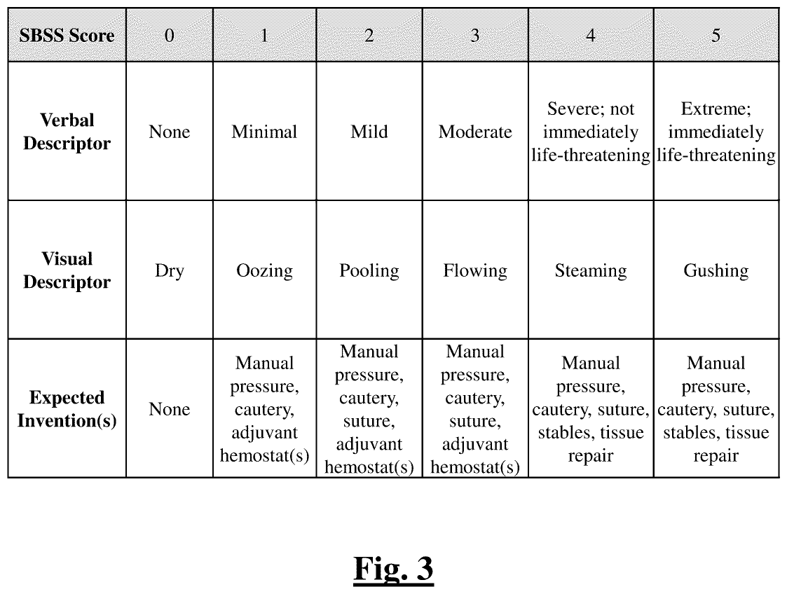

FIG. 3 shows a chart of surface bleeding severity scale (SBSS) scores verbal and visual descriptors and corresponding expected interventions. The SBSS is a validated bleeding scale developed for harmonized assessment of bleeding severity and evaluation of hemostasis. The SBSS consists of 6 subscales of bleeding (0=none, 1=minimal, 2=mild, 3=moderate, 4=severe; not immediately life-threatening, 5=extreme; immediately life threatening). The score has been successfully used to train surgeons in recognizing severities of bleeding related to in-and exclusion criteria in clinical investigations, and for assessment of endpoints of hemostasis for performance assessments. Hemostatic Patch 200 will be used as an adjunct to hemostasis for minimal, mild, moderate bleeding sites, defined by the Surface Bleeding Severity Scale (SBSS) 1-3.

The studies herein use the SBSS as the validated bleeding scale in preclinical evaluations of Hemostatic Patch 200 and in clinical investigations. Furthermore, as SBSS will be considered the predominant validated bleeding scale with which Hemostatic Patch 200 will be determined to be safe and performing as intended, the SBSS will be used in outward facing documents (e.g., Instructions for Use, Summary of Safety and Clinical Performance, etc.,) to delineate the clinical indication of Hemostatic Patch 200.

### Study 1: A Prospective, Multicenter, Single-arm, Clinical Investigation Evaluating the Safety and Performance of Hemostatic Patch 200 for Hemostasis during Open Liver Surgery

A first-in-human clinical investigation was performed at 3 clinical sites in the Netherlands to collect clinical safety and performance data on the use of Hemostatic Patch 200 during internal surgery, specifically liver surgery. The study was conducted in compliance with ISO14155: 2020, the Declaration of Helsinki and any national or local legislations. Written informed consent was obtained from all patients prior to entry into the study. The study was closed in January 2022. This data set was appraised for relevance following MDCG 2020-6 and an overview of the data is provided below as considered relevant to this clinical evaluation. his was a pre-market, prospective, single arm, multicenter, first-in-human clinical investigation. The clinical investigation was split into 2 stages:
Stage I of the clinical investigation was performed with a small cohort of subjects within which the initial safety of Hemostatic Patch 200 was evaluated. A total of 8 subjects were treated in Stage 1, after which the enrollment into the clinical investigation was paused. A Data Monitoring Committee (DMC) reviewed safety in this subset of subjects and decided that the study was allowed to proceed.
Stage II of the clinical investigation enrolled 39 subjects who were treated with Hemostatic Patch 200. Stage II was used to evaluate safety and performance of Hemostatic Patch 200.

In both Stage I and Stage II, subjects followed the same clinical investigation pathway. Stage I subjects were analyzed for safety only, whereas Stage II subjects were analyzed for both safety and performance.

### Primary Objectives and Hypothesis

The objective of the study was to evaluate the clinical safety and performance of Hemostatic Patch 200 in open liver surgery.

It was expected that Hemostatic Patch 200 achieved hemostasis within a specified time frame in the majority of patients and has a good safety profile. The hypothesis was defined as follows: the percentage of cases achieving hemostasis at 3 minutes using Hemostatic Patch 200 is significantly greater than the literature-based performance goal (PG) of 65.4% (i.e., whether Hemostatic Patch 200 is non-inferior compared to the standard of care). Thus, the statistical null (H0) and alternative (H1) hypotheses were the following:
H0: pGATT≤65.4%;
H1: pGATT>65.4%

### Primary Performance Endpoint

The primary performance endpoint was defined as non-inferiority of Hemostatic Patch 200 compared to the standard of care regarding the percentage of cases achieving hemostasis at 3 minutes (i.e., demonstrate that Hemostatic Patch 200 is significantly greater than the literature-based performance goal of 65.4%).

Hemostasis was defined by a grade of 0 (None/Dry) on the SBSS. The SBSS provides a validated score for assessment of bleeding at the target site, and consists of 6 subscales (0=none, 1=minimal, 2=mild, 3=moderate, 4=severe; not immediately life-threatening, 5=extreme; immediately life threatening). Investigators were trained on the assessment scale prior to the investigation to have consistent assessment of bleeding at the target site.

Achievement of hemostasis was verified every 30 seconds, starting from the time that Hemostatic Patch 200 was positioned and pressure was initiated. If hemostasis had not been achieved after 5 minutes of application (SBSS 1-5), then treatment was considered a failure and additional hemostatic agents or techniques could be used.

### Secondary Performance Endpoints

The following secondary endpoints were defined:
Mean time to hemostasis (seconds);
Percentage of hemostasis at 30, 60, 90, 120 and 150 seconds.

There was no formal hypothesis testing performed on the secondary performance endpoints.

### Safety Endpoints

The safety of Hemostatic Patch 200 was assessed by the nature, severity and incidence of device related adverse events. Adverse events may include:
Toxic response
Thromboembolic event
(Re)Bleeding
Allergic reaction
Pain
New surgery
Infection
Blockage of artery or vein/ischemia of organs
Damage of organs and vessels
Pulsatile hematoma
Closing of intestinal track
Biloma
Encapsulated or rolled-up device

No formal hypothesis testing was performed for the safety endpoint of this clinical investigation. The adverse events found for Hemostatic Patch 200 were compared to the current knowledge and state of the art for hemostatic methods in open liver surgery to assess whether the device is associated with acceptable safety outcomes.

### Exploratory Endpoints

In addition to the primary endpoint, the following exploratory endpoints were recorded:
Surgery time (minutes)
Blood loss (mL) during surgery
Blood transfusion (mL) during hospitalization
SBSS (0-5) at the target bleeding site
Use of adjunct hemostatic agents/techniques (e.g. cautery, sutures or staples)
Amount of material needed versus bleeding surfaces
User satisfaction (questionnaire)

### Statistical Analysis

Descriptive statistics were presented for each variable. Continuous variables were summarized using descriptive statistics (number of subjects, mean, median, quartiles, standard deviation, minimum, and maximum). Categorical variables were summarized using frequencies and percentages of subjects in each category.

### Results

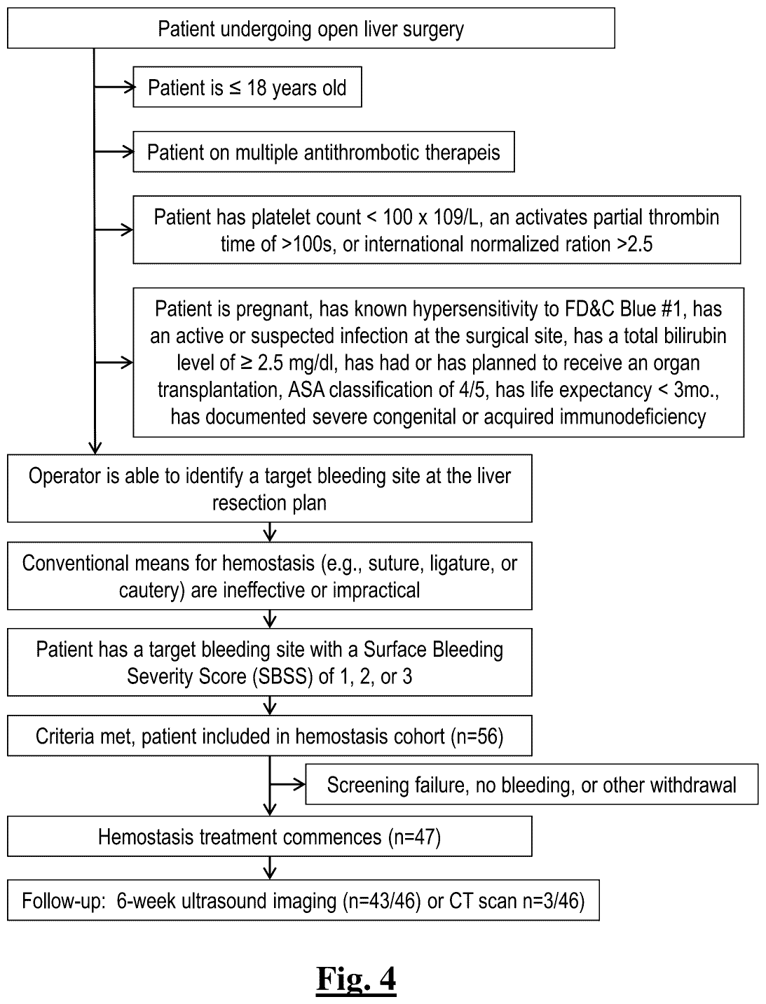

FIG. 4 shows a representative overview of an example of a study flow used for this disclosure. Inclusion criteria included subjects undergoing an elective open liver surgery. Preprocedural inclusion criteria further included a subject (i) scheduled to undergo the elective open surgery on the liver; (ii) willing and able to give written informed consent for investigation participation; (iii) 18 years of age of older at the time of enrollment; and (iv) has been informed of the nature of the clinical investigation. Intraoperative inclusion criteria included a subject in whom the investigator or operator is able to identify a target bleeding site at the liver resection plane for which any applicable conventional means for hemostasis (e.g., suture, ligature, or cautery) are ineffective or impractical and the choice is made to use a topical hemostat for control of hemostasis. Intraoperative inclusion also included a subject that has a target bleeding site with a Surface Bleeding Severity Score (SBSS) of 1, 2, or 3.

A total of 56 subjects were enrolled at 3 investigational centers, of which 8 subjects in Stage I and 39 subjects in Stage II. All 56 subjects provided written informed consent. A total of 9 subjects were withdrawn pre-treatment (2 subjects were a screening failure, 5 subjects did not meet procedure eligibility, 2 subjects were withdrawn for other reasons). The Safety population thus included 47 subjects and the Full Analysis Set (FAS) included 39 subjects. The Per Protocol (PP) defined as FAS subjects who did not have major protocol deviations with data analyzed according to treatment received, included 37 subjects. 97.9% (46/47) of subjects completed the 6-week follow-up visit.

### Demographics

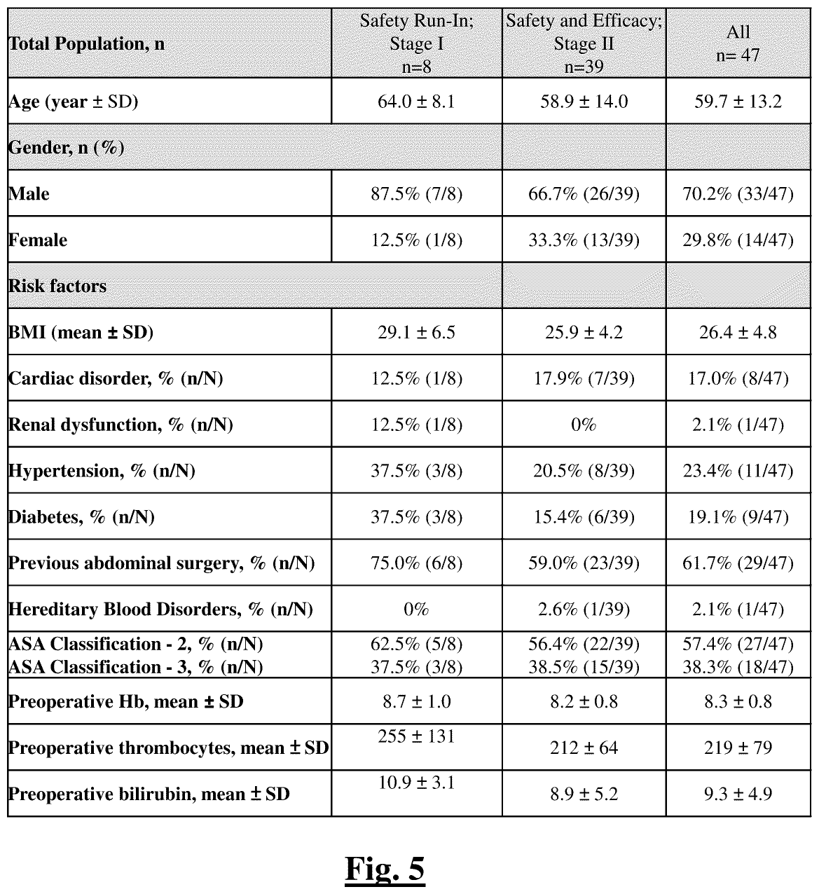

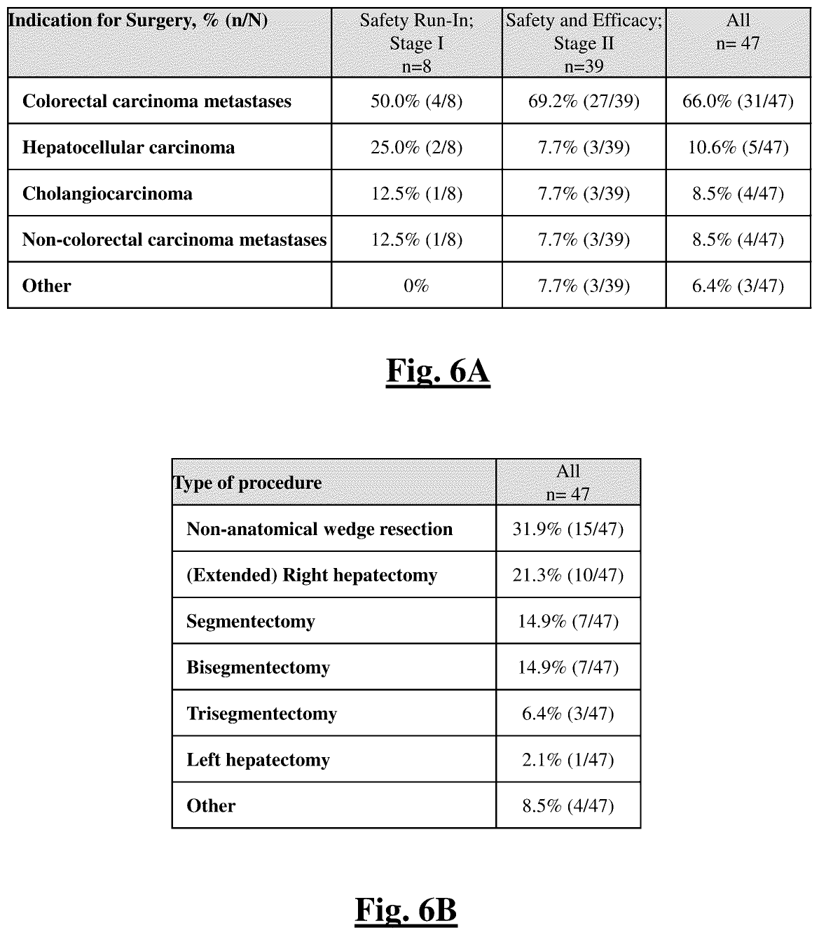

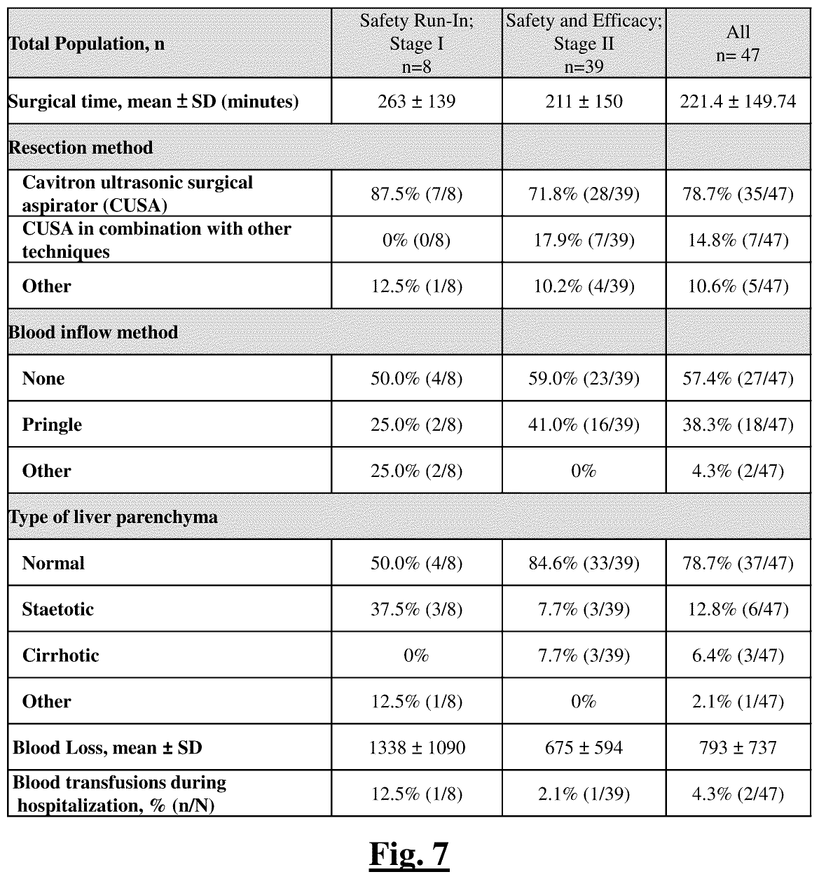

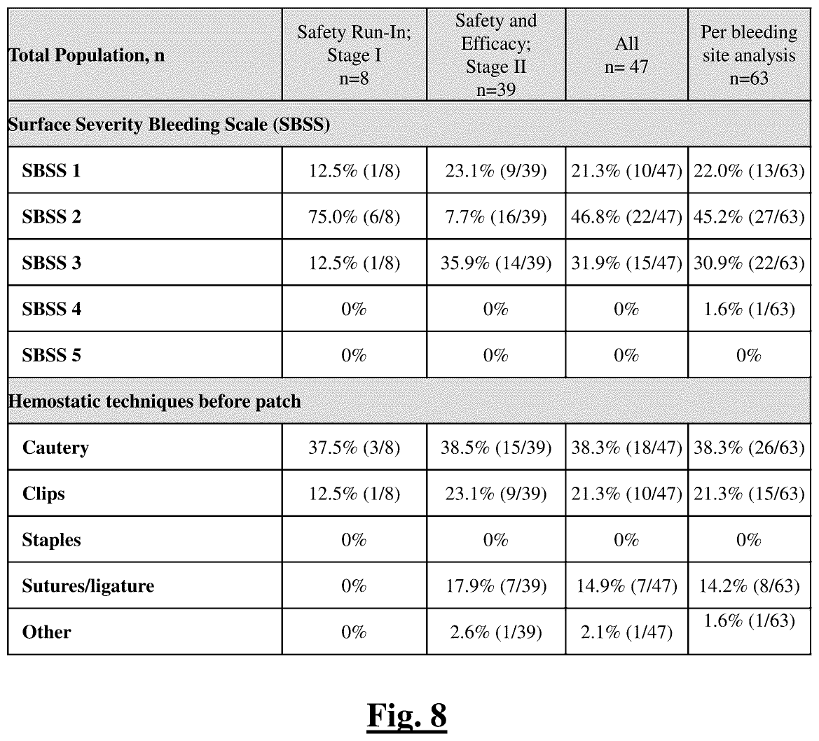

The mean age of included subjects was 59.7±13.2 years (FIG. 5). The majority of included subjects was male (70.2%, 33/47), with origin Europe, Middle East, North Africa (White, 91.5%, 43/47). The disease diagnosis, summarized in FIG. 6A, included colorectal carcinoma metastases (66.0%, 31/47), hepatocellular carcinoma (10.6%, 5/47), cholangiocarcinoma (8.5%, 4/47), non-colorectal carcinoma metastases (8.5%, 4/47) and other diagnosis (6.4%, 3/47). Other diagnoses included: intrahepatic gallstones (n=1), lesion in cirrhotic liver (n=1), and partly cystic/solid lesion (n=1).

The majority of subjects did not use Ascal (93.6% 44/47) or other antithrombotic medication (93.6% 44/47). Allergies to one of the Hemostatic Patch 200 components were largely unknown. Baseline laboratory values were within normal ranges for most subjects.

FIG. 6B is a table summarizing surgical characteristics for the plurality of subjects in the first study of this disclosure. Between both stages, the type of procedure mostly included non-anatomical wedge resection, with 31.9% ( 15/47). Approximately 21.3% underwent a right hepatectomy ( 10/47), while 14.9% underwent a segmentectomy ( 7/47) or a bisegmentectomy ( 7/47). The remaining subjects underwent a trisegmentectomy (6.4%; 3/47), a left hepectomy (2.1%; 1/47), or other (8.5%; 4/47).

### Treatment Details

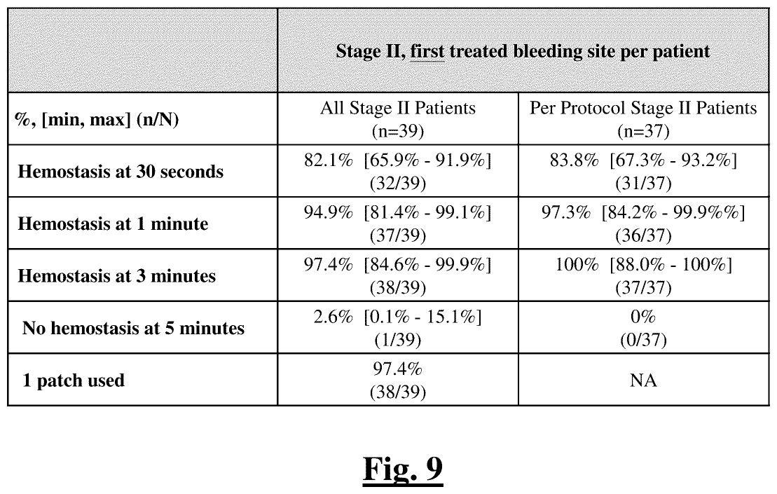

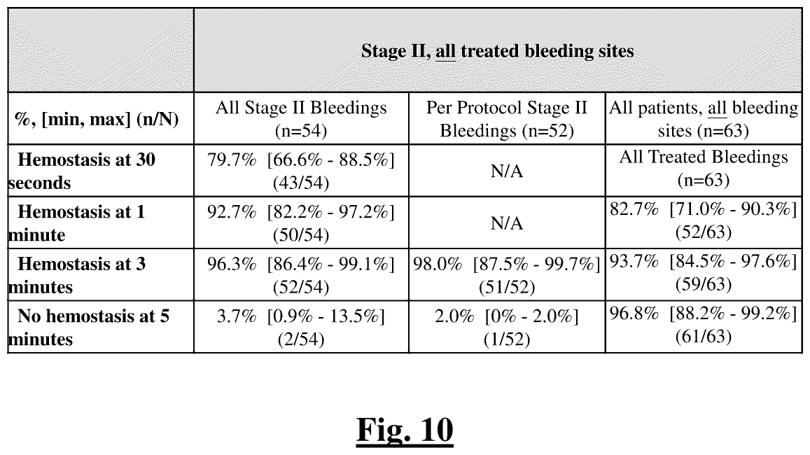

In the FAS Population, the mean surgery time was 221.4±149.74 minutes. FIG. 7 is a table summarizing treatment details for the plurality of subjects in the study described herein. The type of hepatic parenchyma was cirrhotic in 7.7% ( 3/39). A Pringle maneuver was used in 41% ( 16/39) and no other inflow reduction was used in the other patients. The resection method was Cavitron ultrasonic surgical aspiration (CUSA) in 71.8% ( 28/39) and CUSA in combination with bipolar, ligasure, harmonic scalpel and diathermia in the remainder, with exception of one patient that did not receive a resection and Hemostatic Patch 200 was used to stop bleeding for an injury that occurred during Radiofrequency Ablation. The analysis of the use of adjunct hemostatic agents/techniques prior to application of Hemostatic Patch 200 is summarized in FIG. 8, and showed that cautery was used in 15 subjects ( 15/39; 38.5%), clips were used in 9 subjects ( 9/39; 23.1%), sutures/ligature were used in 7 subjects (7/39; 17.9%), and other undefined hemostatic agents/techniques were used in one subject ( 1/39; 2.6%). The SBSS at the target bleeding site was minimal (SBSS 1) in 9 subjects ( 9/39; 22.1%), mild (SBSS 2) in 16 subjects ( 16/39; 41.0%), and moderate (SBSS 3) in 14 subjects ( 14/39; 35.9%). The mean size of Hemostatic Patch 200 applied per cm² bleeding surface was 8.7±10.70 cm².

The mean user satisfaction, as reported by surgeons completing the system usability scale (SUS) score, was 88.4±7.64 (on a scale of 0-100), indicating high user satisfaction.

### Primary Performance Analysis

The primary endpoint of the study was to determine whether Hemostatic Patch 200 is non-inferior compared with standard of care regarding the percentage of patients achieving hemostasis at 3 minutes. The performance goal of 65.4% was based on the weighted average of hemostasis at 3 minutes in 6 RCTs performed with benchmark devices.

FIG. 9 provides a table summarizing hemostasis endpoints for first treated bleeding site per Stage II patient. In approximately 82.1% ( 32/39) of subjects, hemostasis was achieved within 30 seconds of placement of Hemostatic Patch 200 at the bleeding site. Within 1 minute, 94.9% of subjects achieved hemostasis (37/39). In the FAS population, 38 of the 39 patients (97.4%, 95% CI: 84.61, 99.87) achieved hemostasis at 3 minutes using Hemostatic Patch 200 hemostasis at 3 minutes. In the PP population, all patients ( 37/37; 100%, 95% CI: 87.97, 100) achieved hemostasis at 3 minutes. In all but one subject ( 38/39; 97.4%) equal to or less than 1 Hemostatic Patch 200 was applied per bleeding site. In one subject, >1 to 2 Hemostatic Patches 200 were applied per bleeding site. The mean blood loss during surgery was 674.7±594.14 mL. There was one patient that received a blood transfusion during hospitalization (1250.0±0.00 mL).

FIG. 10 provides a table summarizing hemostasis endpoints for all treated bleeding sites (e.g., more than one bleeding site per subject) for Stage II subjects and all patients. In approximately 79.7% ( 43/54) of bleeding sites for Stage II subjects, hemostasis was achieved within 30 seconds of placement of Hemostatic Patch 200 at the bleeding site. Within 1 minute, 92.7% of bleeding sites achieved hemostasis ( 50/54), and within 3 minutes, all but two bleeding sites achieved hemostasis (96.3%; 52/54). In approximately 82.7% ( 52/63) of bleeding sites for all subjects, hemostasis was achieved within 30 seconds of placement of Hemostatic Patch 200 at the bleeding site. Within 1 minute, 93.7% of bleeding sites achieved hemostasis ( 59/63), and within 3 minutes, all but two bleeding sites achieved hemostasis (96.8%; 61/63).

Hemostatic Patch 200 was shown to be statistically non-inferior to standard of care for the achievement of hemostasis at 3 minutes (P<0.001) in both the FAS and PP population. The percentage of bleeding sites that achieved hemostasis at 3 minutes using Hemostatic Patch 200 was 96.3% ( 52/54) in the FAS Population, 98.0% ( 51/52) in the PP Population and 96.7% ( 61/63) in the Safety Population.

### Secondary Endpoint Analysis

The first secondary endpoint was the mean time to hemostasis (in seconds). The mean time to hemostasis was 54.6±107.48 seconds for subjects in the FAS Population and 38.1±26.12 seconds for subjects in the PP Population (not shown). The second secondary endpoint was the percentage of hemostasis at 30, 60, 90, 120 and 150 seconds. In the FAS Population, the percentage of hemostasis was 82.1% ( 32/39) at 30 seconds and 94.9% ( 37/39) at 60, 90, 120, and 150 seconds. One (1) subject in the FAS group failed to show hemostasis at 5 minutes. In the PP Population, the percentage of hemostasis was 83.8% ( 31/37) at 30 seconds and 97.3% ( 36/37) at 60, 90, 120, and 150 seconds. All subjects in the PP Population had achieved hemostasis at 5 minutes.

### Safety Analysis Overall Analysis

The analysis of AEs was performed for the Safety Population. The severity and causality to the investigational device or procedure was assessed. In total, 28 of the 47 subjects (59.6%) experienced AEs and 7 of the 47 subjects (14.9%) experienced SAEs. Most of these subjects experienced AEs that were related to the study procedure (24 subjects, 51.1%). A few subjects experienced AEs that were possibly related to the device (3 subjects, 6.4%); there were no events that were probably related to the device or with a causal relationship with the device.

In the 7 subjects who experienced SAEs, the SAEs were related to the study procedure in 6 subjects (12.8%) and one SAE was also related to the device in one subject (2.1%). The mean number of AEs was 1.6±0.84 in the 28 subjects with AEs. Most subjects experienced no AE (19 subjects; 40.4%) or one AE (17 subjects; 36.2%). The other subjects experienced 2 AEs (7 subjects, 14.9%), 3 AEs (3 subjects, 6.4%), or 4 AEs (1 subject, 2.1%).

There were 44 AEs in total in the 28 subjects with AEs. Most subjects experienced gastrointestinal disorders (5 subjects; 10.6%), hepatobiliary disorders (15 subjects; 31.9%), infections and infestations (8 subjects, 17.0%), and/or AEs related to injury and procedural complications (4 subjects; 8.5%). From the 44 AEs in total, 38 AEs were mild, 3 AEs were moderate, and 3 AEs were severe. From the 28 subjects with AEs, 26 subjects experienced mild AEs (55.3%), 3 subjects experienced moderate AEs (6.4%), and 3 subjects experienced severe AEs (6.4%). The 3 severe AEs involved ascites, hepatic failure, and an abdominal abscess. None of the AEs led to study withdrawal and there were no mortalities during the course of the trial.

In total, 36 of the 44 AEs were related to the procedure. Three (3) AEs were also related to the device. The 3 AEs that were possibly related to the device involved one subject with biloma, one subject with an perihepatic abscess, and one subject with post-procedural hematoma. There were no events that were probably related to the device or with a causal relationship with the device. Of note, all adverse events are also common complications of liver surgery and were, besides possibly related to the device, also marked as probably related to the procedure (e.g., the abscess and biloma) or with a causal relationship to the procedure (e.g., the hematoma).

One device-related AE, involving an perihepatic abscess, was also considered a SAE. The event involved a subject who underwent a CT scan because of pain in the upper belly. The CT scan showed an abscess around the liver. The patient was re-hospitalized and radiological drainage of the abscess was performed; therefore, the event was a SAE. No micro-organisms were found in the fluid. The event resolved without sequela after 4 days. This event was possibly related to the device because exclusion of a relationship could not be established.

### Follow-Up Data: Ultrasound Imaging

At the 6-week follow-up visit, with a mean of 44.8±8.57 days following surgery, subjects underwent imaging examination of the resection area to identify any device encapsulation, rolled-up device, or evidence of a biloma or pseudo-aneurysm. In most subjects ( 43/46; 93.5%) the imaging method was ultrasound imaging per protocol; the other subjects ( 3/46; 6.5%) underwent imaging using a CT scan as these were clinically indicated. The imaging results indicated no subjects with device encapsulation, rolled-up device, or evidence of pseudo-aneurysm ( 0/46; 0.0%).

For all subjects with imaging results (n=46), which includes both the subjects with a clinically indicated CT scan (n=3) and routine ultrasound imaging (n=43), the answer on the question “Evidence of biloma”, based on imaging result, was given as “yes” in 10 subjects ( 10/46; 21.7%). Next to the bilomas diagnosed in 3 subjects based on the clinically indicated CT scans, there were 7 subjects ( 7/43; 16.3%) with evidence of biloma as indicated on the routine ultrasound imaging performed at 6 weeks. Following further analysis, the imaging bilomas were evaluated as possible biloma (n=3), suspicion of partly biloma (n=1), fluid collection being seroma or biloma (n=1), fluid collection being hematoma/biloma (n=1), and biloma (n=1). The 6-week routine ultrasound imaging further indicated that there were 4 subjects with evidence of a hematoma ( 4/43; 9.3%). Following further analysis, the imaging hematomas were evaluated as fluid collection with partly hematoma (n=1), fluid collection, probably hematoma (n=1), small hematoma (n=1), and encapsulation maybe hematoma (n=1). All events were categorized according to the worst-case event that was mentioned in the imaging report, but all events were considered to be mild adverse events and none of these events required any further medical intervention. None of these events were confirmed as biloma or hematoma by percutaneous or operative drainage, and thus, were most likely minor sterile fluid collections.

### Usability Questionnaire

The mean user satisfaction, as reported by surgeons completing the system usability scale (SUS) score, was 86.2±9.80 (on a scale of 0-100), indicating high user satisfaction. The results of the user satisfaction questionnaire also indicate that users were satisfied with Hemostatic Patch 200. From all 27 questions of the user satisfaction questionnaire regarding medical devices specific, 24 questions were answered with a neutral, positive or very positive reply in all users ( 47/47; 100%). The other 3 questions were answered with a neutral, positive or very positive reply in 91.5% ( 43/47)-97.7% ( 46/47) of the users. From all 10 questions of the user satisfaction questionnaire regarding SUS, 9 questions were answered with a neutral, positive, or very positive reply in all users ( 47/47; 100%) and one question was answered with a neutral, positive, or very positive reply in most users ( 44/47; 93.6%).

### Conclusion

The initial first-in-human clinical investigation on Hemostatic Patch 200 was conducted in accordance with ISO14155: 2020 and complied with the regional and ethical principles that have their origin in the Declaration of Helsinki and recommendations guiding physicians in biomedical research involving human subjects adopted by the 18th World Medical Assembly, Helsinki, Finland, 1964 and later revisions. The design of the study (e.g., follow-up time, patient population) is deemed sufficient to assess the safety and performance of Hemostatic Patch 200 for the intended use as provided in the IFU, specifically for liver surgery. In accordance with the appraisal criteria in the Literature Review Protocol (LRP), the data of a prospective cohort (i.e., non-randomized, prospective; Level II), the study is considered of acceptable quality.

Most patients were approximately 60 years of age, 30% were female, and 91.5% were white/Caucasian. The indication for surgery was colorectal metastases in 66% of patients and 6.4% of patients had liver cirrhosis. The population as included in this trial represents a similar population as seen in other hemostatic trials in liver surgery.

The primary performance endpoint was defined as non-inferiority of Hemostatic Patch 200 compared to the standard of care regarding the percentage of subjects achieving hemostasis at 3 minutes. Hemostatic Patch 200 was shown to be statistically non-inferior to standard of care for the achievement of hemostasis at 3 minutes in both the FAS and PP Population. The percentage of subjects that achieved hemostasis at 3 minutes using Hemostatic Patch 200 was 97.4% in the FAS Population and 100% in the PP Population. This rate of hemostasis is significantly higher than the literature-based performance goal of 65.4%. Analyses of the secondary endpoints showed that the mean time to hemostasis was 54.6±107.48 seconds for subjects in the FAS Population and 38.1±26.12 seconds for subjects in the PP Population. Almost all subjects (94.9% in the FAS Population and 97.3% in the PP Population) had achieved hemostasis at 60 seconds. In the PP Population, all subjects had achieved hemostasis at 5 minutes. In the FAS Population, there was one subject who did not achieve hemostasis with the first application of Hemostatic Patch 200. The subject did achieve hemostasis initially, but a hematoma was observed within 5 minutes. The hematoma was removed per instructions for use of Hemostatic Patch 200, and an additional piece of Hemostatic Patch 200 was applied, after which hemostasis was achieved.

The safety of Hemostatic Patch 200 was assessed by the nature, severity and incidence of device related AEs in the Safety Population. In total, 28 of the 47 subjects (46.8%) experienced AEs and 7 of the 47 subjects (14.9%) experienced SAEs. There were 44 AEs in total, of which 36 AEs were related to the procedure and 3 of these AEs were also related to the device. There were 7 SAEs in total, of which 6 SAEs were related to the procedure and one SAEs was also related to the device. This finding is in line with the literature regarding benchmark devices, showing that 42-100% of subjects experience at least one AE during liver resection surgery. Moreover, these studies show that most AEs are primarily associated with procedural complications and are not related to the hemostatic product used, which is similar to the findings in the current study. With Hemostatic Patch 200, the incidence of device related AEs was 6.4%, which is considered below the acceptance criterion for safety (≤7.3%). Device-related AEs included biloma (n=1), perihepatic abscess (n=1), and post-procedural hematoma (n=1); all of which were adjudicated conservatively according to the principle of worst-case where it could not be excluded that there was a relationship with the device. The reported AEs for Hemostatic Patch 200 are in line with the device related AEs reported for benchmark devices, as these include the risk of bile leak, hematoma, localized intra-abdominal fluid collection, peritoneal abscess, liver abscess, and postoperative abscess, among other risks, as expected in the setting of major abdominal surgery.

At 6 weeks follow-up, a routine ultrasound of the liver was performed. Three (3) subjects underwent a CT scan because of clinical reasons and were diagnosed with biloma ( 3/47; 6.4%) and an abscess ( 1/47; 2.1%). The results of the other 43 patients undergoing routine ultrasound imaging indicated that there was evidence of biloma in 7 of the subjects ( 7/43; 16.3%), so that overall 10 subjects ( 10/47; 21.3%) were given the diagnosis of biloma. This finding on imaging was typically reported as perihepatic fluid being visible which could be a biloma, but without confirmation that the fluid actually was bile. None of these events were confirmed as biloma by percutaneous or operative drainage, and thus, were most likely minor sterile fluid collections. These events of fluid were reported as biloma according to the worst-case principle where if there was suspicion of a biloma, it was reported as such. Moreover, no medical action was required based on the routine imaging and none of these events resulted in any clinical sequalae. There were 3 patients ( 3/47; 6.4%) that presented with a biloma based on the clinical presentation. It should be noted that bile leakage is a common complication of liver resection surgery. In fact, bile leakage is one of the most frequent complications after liver resection surgery. Even in randomized trials on the use of sealant products to reduce postoperative bile leakage, it is a frequent event.

In the current clinical investigation, Hemostatic Patch 200 was used as a hemostatic device, and therefore applied only on a target bleeding site and not the overall resected area and bile leakage could have occurred from the exposed resection area not covered by Hemostatic Patch 200. The confirmed clinical rate of bile leakage occurring in this study (6.4%) is consistent with the published literature. The majority of the biloma-related events were considered clinically insignificant and required no intervention and can be considered to be purely imaging findings, and were most likely minor sterile fluid collections as it was not confirmed by percutaneous or surgical exploration that the fluid was actually bile. Indeed, in previous clinical trials where routine imaging was performed after liver surgery, similar to the protocol of the current trial with a scheduled ultrasound, postoperative CT scans identified that 27% of patients had a fluid collection of 100 mL or more at the resection surface. The meta-analysis performed to set an acceptance criteria for fluid collections found on imaging, reported that a mean of 18.0% (95% CI: 6.7%, 40.1%) of patients had fluid collections, and with the investigation on Hemostatic Patch 200 demonstrating a rate of 21.7%, this is acceptable and Hemostatic Patch 200 can be considered safe.

This clinical investigation presents the first-in-human results for Hemostatic Patch 200. The safety and performance of Hemostatic Patch 200 was evaluated in adult subjects undergoing elective open liver surgery. The clinical data indicate that Hemostatic Patch 200 is safe and effective for use in open liver surgery in adult subjects. The performance acceptance criterion of achieving hemostasis ≥65.4% was met, and the performance of Hemostatic Patch 200 (97.4%) was considered comparable and significantly higher when compared to benchmark devices. Application of Hemostatic Patch 200 further did not give raise to any new or unique safety concerns. The incidence (≤7.3%) and type of AEs related to the use of Hemostatic Patch 200 were comparable to AEs related to the use of benchmark devices as described in the state-of-the-art literature, which demonstrates that Hemostatic Patch 200 is associated with acceptable safety outcomes.

### Study 2: Results of non-GLP Hemostatic Patch 200 versus Comparative Device 1 in a Porcine Kidney Bleeding Model

The aim of this study was to evaluate the acute hemostatic performance of Hemostatic Patch 200 versus the standard of care, Comparative Device 1 (FloSeal 5 mL; Baxter Healthcare, Deerfield, Illinois, United States) in a porcine kidney severe cavity bleeding model, but modified with the use of heparinization to represent medication-induced or in-use coagulopathic conditions.

Four pigs underwent surgery on their kidneys, with heparinization performed with a targeted active clotting time (ACT) 1.5-2.5 times higher than baseline. In each kidney, 6 reproducible lesions were created (i.e., 3 on the anterior and 3 on the posterior surface) with a 10 mm diameter biopsy punch and a depth of approximately 10 mm; the punched tissue was cut away using scissors. Alternating treatments were subsequently initiated either with Comparative Device 1 or Hemostatic Patch 200 (GATT-Patch, 10×5 cm; GATT Technologies BV, Nijmegen, The Netherlands) so that each kidney ultimately had 3 applications of Comparative Device 1 and 3 of Hemostatic Patch 200.

Comparative Device 1 was used per the instructions for use: the cavity was filled and 2 minutes of pressure with a saline-wetted gauze was initiated. Hemostasis was checked at 2 minutes after careful removal of the gauze to not disturb and disrupt the hemostatic matrix. If bleeding persisted, additional Comparative Device 1 was applied and again followed by 2 minutes of pressure with a saline-wetted gauze. This procedure was repeated for a max of 3 Comparative Device 1 applications: if bleeding persisted after 3 applications, the treatment was a failure. If at any time hemostasis was achieved and maintained to 8 minutes, the treatment was a pass.

For the lesions treated with Hemostatic Patch 200, about a 3-3.5×3-3.5 cm piece of the patch was packed into the cavity with immediate topical application of a piece of the patch that was cut to a round shape with a diameter of about 4 cm, on top of the bleeding so that it overlapped non-bleeding tissue by at least 1 cm on all sides, according to the IFU. Thirty seconds of pressure with a saline-wetted gauze was initiated, after which hemostasis was checked by careful removal of the gauze. If bleeding persisted, an additional 30 seconds of pressure was applied. If bleeding persisted again, Hemostatic Patch 200 was removed and a second treatment using the same approach was applied. If at any time hemostasis was achieved and maintained to 8 minutes, treatment was a pass.

Time to hemostasis for both Comparative Device 1 and Hemostatic Patch 200 was considered to be the latest time when hemostasis was achieved and maintained to 8 minutes from the initial application. The bleeding severity was adjudicated according to the Severity Bleeding Surface Scale by two trained investigators that hold a certificate on the adjudication of bleeding on this scale of 0-5 bleeding severity.

A total of 48 lesions were created, treated with Hemostatic Patch 200 (n=25) and Comparative Device 1 (n=23). The imbalance in number of lesions per treatment group was caused by the unavailability of Comparative Device 1 at the end of one of the procedures, where the lesion was alternatively treated with Hemostatic Patch 200. The majority of bleedings were severe or life-threatening (n=43; 90%), with the remainder being moderate (n=5; 10%). The heparinization protocol resulted in a prolonged ACT value that was conform the targeted 1.5 to 2.5 times baseline for all treatment applications.

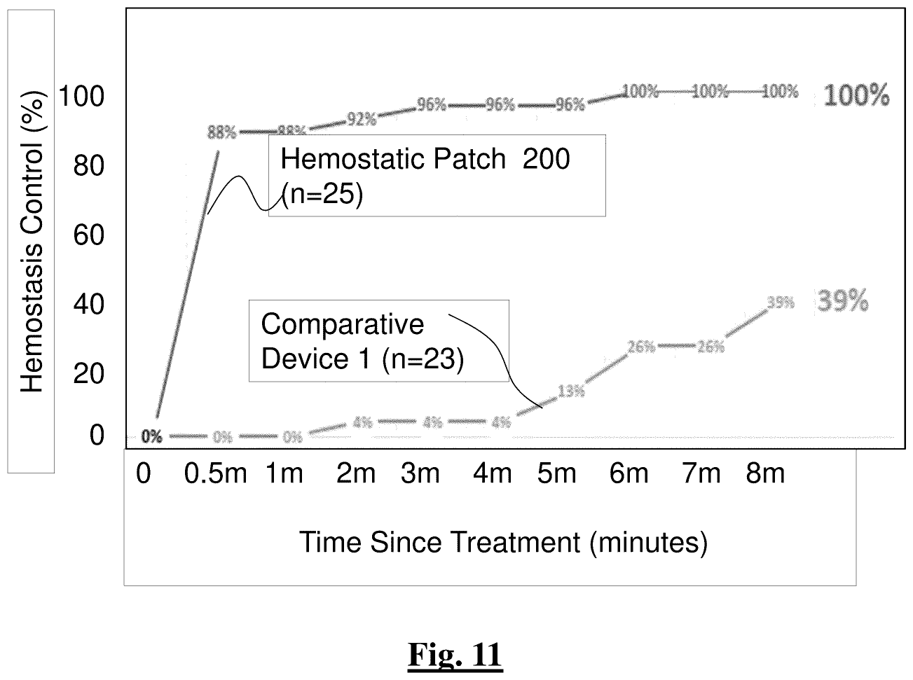

Of the 23 bleedings treated with Comparative Device 1 and 25 bleedings treated with Hemostatic Patch 200, hemostasis at 8 minutes was 39% versus 100%, respectively. As shown in FIG. 11, hemostasis after the initial IFU-indicated application time of 2 minutes for Comparative Device 1 and 30 seconds for Hemostatic Patch 200 was respectively 4% (n= 1/23) versus 88% (n= 21/25). A second application of Comparative Device 1 was needed in 96% (n= 22/23) and resulted in an additional 9% ( 2/23) successful hemostasis rate, while a third application was required in 87% (n=20) ultimately leading to a 39% hemostatic success rate at 8 minutes. After Hemostatic Patch 200 application, an additional 30 seconds of pressure was performed in 4% (n= 1/25), resulting in a 92% successful hemostasis rate. Again an additional 30 seconds of pressure was performed in 4% (n= 1/25) resulting in a 96% successful hemostasis rate at 3 minutes. In only one case there was a need to remove the initial Hemostatic Patch 200 application and perform a new application, which resulted in successful hemostasis before 6 minutes.

Hemostasis of severe kidney cavity bleedings can quickly and reliably be achieved with Hemostatic Patch 200, while Comparative Device 1 seems unable to present as a solution in these challenging cases. This data provides evidence on several important aspects of Hemostatic Patch 200:
1. The performance of Hemostatic Patch 200 is irrespective of anticoagulation use causing coagulopathy, as high hemostatic performance was achieved with Hemostatic Patch 200 while all treated bleedings in this study were under heparinized conditions;
2. The performance of Hemostatic Patch 200 on the kidney is strong, demonstrating its ability to be used in a wide range of clinical scenarios, when viewed in addition to the other studies already performed with Hemostatic Patch 200 further elaborated upon in this document;
3. As compared with the standard of care of flowable hemostatic matrices for treatment of highly irregular tissue surfaces, here represented by cavity lesions, Hemostatic Patch 200 presents as a superior option due to its flexibility and pliability;
4. While the initial indication for Hemostatic Patch 200 is proposed to be for use on minimal, mild, and moderate bleeding, this study supports use of Hemostatic Patch 200 in severe bleedings for potential future regulatory approvals; and
5. The 30-seconds application time of Hemostatic Patch 200, as described in its IFU, is adequate in achieving hemostasis in the vast majority of cases (e.g. 88%), similar to what is achieved in other preclinical and clinical studies further elaborated upon in this document.

### Study 3: Ex Vivo Porcine Liver Perfusion Model with Whole Blood to Compare Hemostatic Patch 200 to Comparative Device 2 and Comparative Device 3

An ex vivo porcine liver perfusion model with whole blood was primarily developed to evaluate hemostatic agents (Hemostatic Patch 200, hemostatic patch prototypes, and other competitive hemostats) on surgical liver bleeding without the need for intensive animal studies.

To validate the ex vivo model, livers and blood were obtained from sacrificed pigs. Ten (10) liters of blood was collected and a standard extracorporeal organ system (ECOPS, Organ Assist, Groningen, the Netherlands) was filled and primed with the blood to circulate through bypass. Fresh heparinized blood was utilized to mimic in-vivo conditions as closely as possible. Five (5) ex-vivo liver perfusion procedures were performed for validation of the model. Livers were mounted onto a perfusion machine and oxygenation, pH, temperature, and blood pressure were kept within-vivo boundaries. Blood was circulated through the liver at a rate of 500 ml/min and a pressure of 5-10 mmHg. Standard punch lesions were then made to the livers to observe hemostasis with 2 commonly used patches: Comparative Device 2 (TachoSil; Corza Medical, Westwood, MA, United States) and Comparative Device 3 (Veriset; Medtronic plc, Dublin, Ireland). In addition, an in vivo heparinized experiment was conducted to compare the efficacy of hemostatic patches for liver lesions similar to the ex vivo model.

Initial testing demonstrated that 3 perfusions could be continued for 4 hours and two (2) for 3.5 hours. In all livers, color and temperature had returned to about normal within 30 minutes after start of the perfusion. When hemostatic performance of Comparative Devices 2 and 3 was compared between use in the ex-vivo liver perfusion model and an in-vivo heparinized porcine model, there was no significant differences in the percentage of applications that resulted in successful hemostasis within 3 minutes. Furthermore, relevant blood gas measurements, coagulation parameters (partial thromboplastin time, prothrombin time, and fibrinogen), as well as clotting time and clot formation time obtained with the ex vivo model, were shown to be comparable to the in vivo measurements and remain constant over 4 hours. Although there were some observed differences between the in vivo and ex vivo model, such as the glucose and erythrocyte measurements, these differences were determined not to affect the evaluation of local hemostatic products for up to 4 hours. Therefore, this validation testing demonstrated that the ex-vivo model can be used for 4 hours with consistent coagulation parameters that are comparable to an in-vivo heparinized porcine model.

This model validation testing established the following test parameters for future use of the ex vivo model: Two (2) livers and 10 liters of heparinized blood (5000 units/L) are collected at the slaughterhouse. Livers are transported on ice and blood is transported at ambient temperature. Within 2 hours after collection, livers are inspected for lesions which are closed with gloves and cyanoacrylate glue. Perfusion parameters are: flow 600 ml/min; pressure 10-12 mmHg; temperature of the blood 37° C. (±1° C.); carbogen 0.25 liters a minute. After checks of color and temperature, product testing can take place.

When comparing hemostatic patches under clinical use to the ex-vivo model, the rate of success is lower on the challenging ex-vivo model. For example, for Comparative Device 2, which is indicated as an adjunct to hemostasis during liver surgery, the pooled results from 3 randomized clinical trials demonstrated that 174 of 233 subjects (74.7%) in the Comparative Device 2 treatment group achieved hemostasis at 3 minutes.

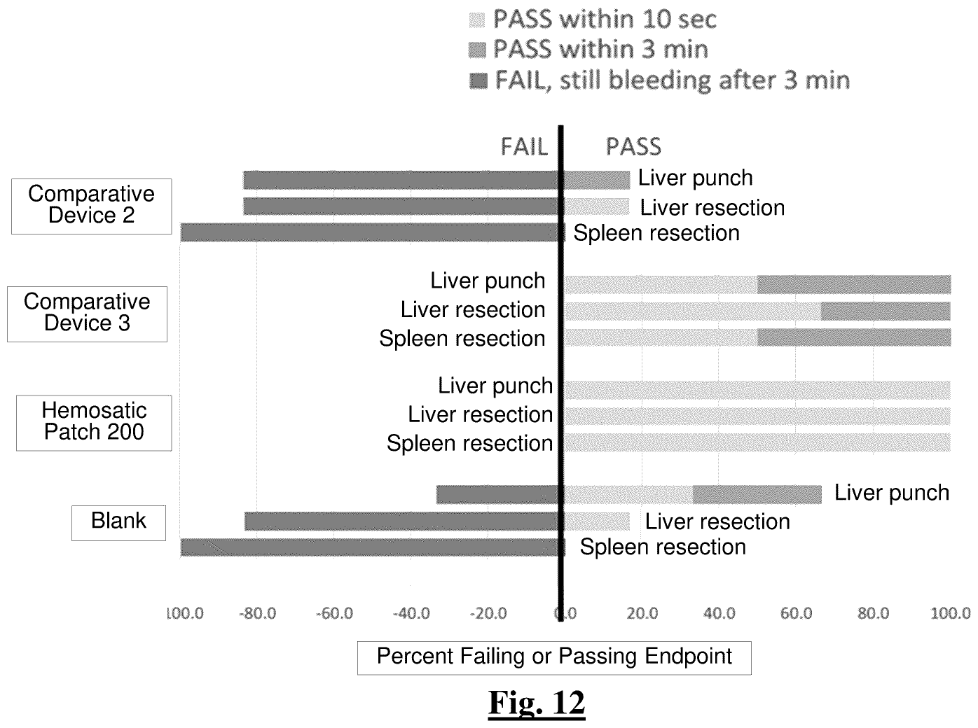

FIG. 12 is a graphical depiction of efficacy of adjusted hemostatic patch compared to a blank, Comparative Device 1, and Comparative Device 2 after a liver punch, liver resection, and spleen resection. As shown, Hemostatic Patch 200 reached hemostasis within 10 seconds pressure of all punch and resection bleedings (light gray), with persistent hemostasis at three minutes. Comparative Device 3 application was successful within three minutes in all cases, however, in 50% of liver punch bleedings, 67% of liver resections, and 50% of spleen resection within 10 seconds. Comparative Device 2 and GFC blank application was successful in only 18% and 67% of punch bleedings at three minutes (0% and 33% at 10 seconds), 18% and 18% of liver resections at three minutes (18% and 18% at three minutes) and 0% and 0% in spleen resections at three minutes. Considering all liver and spleen punctures and resections, Hemostatic Patch 200 versus Comparative Device 3, Comparative Device 2 and GFC-Blank reached hemostasis in 10 seconds in 100%, 42.8%, 7.1% and 14.3% respectively, and at 3 minutes in 100%, 100%, 14.3% and 35.7% respectively.

### Study 4: GLP Study Hemostatic Patch 200 Compared to Comparative Device 4 and Comparative Device 5—Evaluation of Efficacy and Safety Following Functional Application on Bleeding Organs in a Swine Model

Twenty-six (26) female swine were enrolled and successfully underwent the implant procedure. Nine (9) were assigned to the 72 hour, eight (8) to the 4 week, and nine (9) to the 8 week cohort. Eight (8) hepatic bleed sites were created per animal for a total of 208 bleeding sites, and no sites were excluded based on bleed severity scores. One hundred thirteen (113) sites (54.3%) were scored with a bleed severity of 2 or 3 (mild-moderate) and 90 sites (43.3%) with a bleed severity of 1 (minimal). Five (5) sites (2.4%) were scored as bleed severity of 0.5. These bleed severity scores correspond to the company's validated Surface Bleeding Severity Scale (SBSS). Specifically, 113 sites demonstrated an SBSS of 2-3 and 95 sites demonstrated an SBSS of 1.

In-vivo lobal placement was made to the organs to observe hemostasis with 2 commonly used hemostats: Comparative Device 4 (Surgifoam, Johnson and Johnson Ethicon, Bridgewater, NJ, United States), where topical RECOTHROM Thrombin was used in combination with every application of Comparative Device 4, and Comparative Device 5 (Hemopatch, Baxter International Inc., Deerfield, IL, United States).

There was one adverse event noted during the implant procedures, but it was unrelated to the application or use of the control article and occurred subsequent to surgeon error. Although the inferior vena cava was nicked with a surgical instrument, it was repaired successfully and the evaluation of articles completed, without impact to study data or animal health. For overall article application, 80 sites were treated with Hemostatic Patch 200 (10 animals), 80 sites treated with Comparative Device 4 (10 animals) and 47 sites treated with Comparative Device 5 (6 animals).

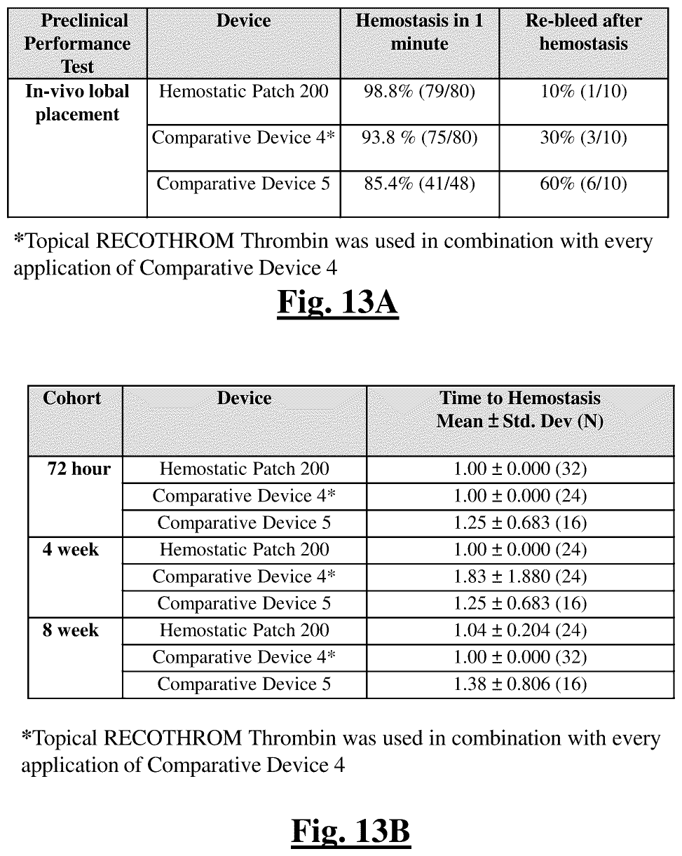

For Hemostatic Patch 200, time to hemostasis was 1 minute for 79 of 80 sites (98.8%); for Comparative Device 4, time to hemostasis was 1 minute for 75 of 80 sites (93.8%); and for Comparative Device 5, time to hemostasis was 1 minute for 41 of 48 sites (85.4%). The average time to hemostasis for each implant is summarized in FIGS. 13A and 13B.

In total, there were 10 re-bleeds after hemostasis was achieved, 6 with Comparative Device 5, 3 with Comparative Device 4 and 1 for Hemostatic Patch 200. There was no evidence of re-bleeding at 30 minutes (±5 minutes) post application for any of the injury sites.

In summary, performance of the Hemostatic Patch 200 successfully demonstrated non-inferiority to the Comparative Device 4 and Comparative Device 5 hemostasis devices when comparing time to hemostasis and re-bleed events at implant and termination in the 72 hour, 4 week and 8 week cohorts.

Material swelling at 30 minutes (±5 minutes) after treatment was similar between hemostatic patches 200 and Comparative Device 5 with scores of ‘0,’ swelling thinner than initial thickness (100% of Hemostatic Patch 200; 83% of Comparative Device 5; 7.5% Comparative Device 4). The remaining 17% of Comparative Device 5 sites were considered a score of ‘1’ (no swelling). For Comparative Device 4, 90% scored a ‘1’ (no swelling) and was also the only article that had a score of ‘2’ in 2.5% of applications (2 of 80 sites), exhibiting minor swelling.

The prevalence of gross adhesions were of similar incidence across both Hemostatic Patch 200 (81%) and Comparative Device 4 (86%) at 72 hours, and somewhat less notable with Comparative Device 5 (50%). At both 4 and 8 weeks, gross adhesions were present across all Test and Control Articles, with distribution indicative secondary effects from the surgical procedure. Clinical pathology data did not present any evidence of active intravascular hemolysis or alterations of coagulation associated with the use of Hemostatic Patch 200.

Histologically, there were no discernable adhesions noted at any of the liver treatment sites across all test and control articles at 72 hours or 4 weeks. At 4 weeks, there was evidence of adhesion of Hemostatic Patch 200 in 17 adjacent diaphragm sites, and evidence of adjacent adhesion of Comparative Device 4 in 17 diaphragm sites. Comparative Device 5 did not present with any microscopic findings of adhesions at 4 weeks. At 8 weeks, there was no evidence of any adhesions at any liver treatment site for all test and control articles, with one exception (histological score of ‘1’).

Finally, test article migration was not observed with Hemostatic Patch 200 in any cohort.

Ultimately, there were no safety concerns of Hemostatic Patch 200 as compared to controls with gross evaluation at necropsy. Evaluation of local tissue effects at all time points after treatment demonstrated that Hemostatic Patch 200 exhibited histological responses consistent with acceptable biocompatibility. Mean reactivity scores of Hemostatic Patch 200 at all time-points after treatment were lower than both Comparative Device 4 and Comparative Device 5. Controls supporting a conclusion of no reactivity of Hemostatic Patch 200 relative to either Control Article. Mineralization was not part of the histological response for Hemostatic Patch 200 at any time point and subsequent to the 72 cohort, there was no material remaining at 4 weeks or 8 weeks after treatment, indicating complete bioresorption by 4 weeks. Finally, histological evaluation of non-target organs were unremarkable suggesting no systemic toxic effects from either Hemostatic Patch 200 or Comparative Device 5 and Comparative Device 4.

### Study 5: Comparison of GLP Evaluation of Efficacy and Safety of the Hemostatic Patch 200 in Open Implant Study and Laparoscopic Application of Bleeding Sites in a Swine Model

To assess the use of Hemostatic Patch 200 in open GLP study, a GLP preclinical study was undertaken to evaluate the effectiveness and safety of Hemostatic Patch 200. This study aimed to demonstrate that Hemostatic Patch 200 performs as intended in the open surgery setting by assessing hemostatic performance, rebleeding rates, safety, and degradation, and comparing these with the minimally invasive surgery (MIS) study results.

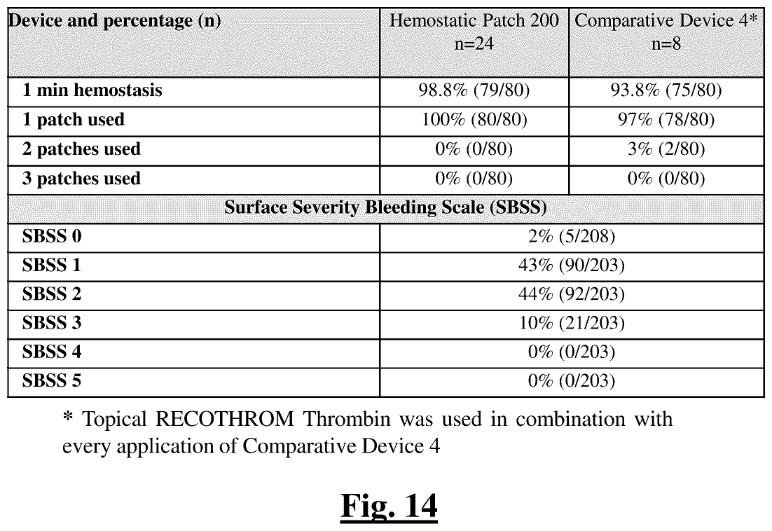

In both the open and laparoscopic GLP study, baseline bleeding severity was distributed across the range of minimal, mild, and moderate bleeding, and this distribution was similar, as shown in FIG. 14.

Four healthy porcine were successfully utilized on this study. A total of 24 Test article sites and 8 Control Article sites were treated. Two bleeding sites were created but not treated/assessed due to high baseline SBSS score (4). These sites were treated with a combination of cautery, which was insufficient to reduce the bleeding, and to stop the bleeding when it was still an SBSS of 4. Regarding pre-treatment SBSS scores for test article treatments, 37.5% (9 of 24) had a score of “1”, 45.8% (11 of 24) had a score of “2” and 16.7% (4 of 24) had a score of “3”. Regarding pre-treatment SBSS scores for control article treatments, 25.0% (2 of 8) had a score of “1”, 37.5% (3 of 8) had a score of “2” and 37.5% (3 of 8) had a score of “3”. All test and control article treatments were hemostatic, SBSS score of “0” at the 30-40 second post-treatment bleeding assessment and all test and control article treatments maintained hemostasis throughout the five-minute post-treatment assessment period, with the exception of test article treatment of lesion 3. For this animal, a SBSS score of “1” was observed at the 3 minute assessment, however the lesion was found to be hemostatic at the 5 minute assessment without any additional pressure time or additional product; it seems likely that this therefore represents a data collection error.

### Endpoint 1: Overall Animal Health (Moribundity)

Overall animal health (moribundity), defined as overall animal health, was assessed through review of physical examinations, clinical observations, clinical pathology, and medical treatments.

The success criteria established for Endpoint 1 was that there would be no clinically significant adverse event leading to early death or mortality due to treatment with the test articles. All assessments suggested that animals remained in good general health throughout the duration of the study. There were no deaths or major adverse events that affected animal health or welfare.

### Endpoint 2: Study Article Handling and Performance

Study article handling and performance was assessed by the Study Surgeon according to four parameters. The four parameters evaluated were: (1) Introduction through a trocar: acceptable if introduced in the body without sticking to the trocar, to the extent it can no longer be used on the bleeding site, (2) Navigation to bleeding site: acceptable if it doesn't stick to other organs/areas to the extent it can no longer be used on the bleeding site, while moving to the bleeding site, (3) Preparation for application: acceptable if patch can be unfolded and positioned at bleeding site and (4) Application: acceptable if placed on bleeding site and pressure applied with wet gauze. There were no success criteria established for Endpoint 2. However, the surgeon response to all criteria were positive, denoted as “acceptable” for both the test article (n=3) and control article (n=1) treated animals.

### Endpoint 3: Study Article Migration

Study article migration was assessed by visual migration and histopathology via light microscopy. Visual migration was assessed qualitatively by the assistant surgeon at the day 3 follow-up procedure and at the termination procedure. Histopathology, performed by the study pathologist, assessed sites on the liver and other tissue away from study article application for any indication of the study article migration.

No success criteria were established for the visual migration assessment, however, in summary there was no indication of test article (n=24) or control article (n=8) migration from any treatment sites at the day 3 and termination procedures. The success criteria regarding histopathology assessment of study article migration was no presence of test article found at non-treated sites collected for histology. Histopathology found no evidence of migrated study article in untreated liver sections.

### Endpoint 4: Hemostatic Success

Hemostatic success was assessed through evaluation of bleeding sites using the Surface Bleeding Severity Scale (SBSS) prior to treatment, post-treatment, and at termination. Also, hemostatic success was evaluated using visual assessment for re-bleeding at the end of treatment (prior to closure), at the day 3 follow-up procedure and at termination. The success criteria for endpoint 4 was that hemostasis (SBSS score of “0”) following application of the test article would be demonstrated to be effective without evidence of re-bleeding at termination. The SBSS scores prior to test and control article treatment ranged from 1-3. All test article treatments (n=24) and control article treatments (n=8) achieved hemostasis by the 5-minute post-treatment SBSS evaluation, with 16.7% of Hemostatic Patch 200 and 37.5% with control requiring additional product(s) to be placed for hemostasis, and there was no evidence of re-bleeding after the 30-minute return to normal intra-abdominal pressures on the day of treatment. Additionally, there was no evidence of re-bleeding at either the day 3 or termination procedures. Therefore, the success criteria for this endpoint was met.

### Endpoint 5: Time to Hemostasis

Time to hemostasis was defined as the time where a SBSS score of “0” was achieved and maintained through the post-treatment evaluation period. Bleeding was evaluated at each treatment site using the SBSS at 30 seconds, 1 minute, 3 minutes and 5 minutes after treatment with the test or control article. In instances where additional test/control article applications were required, the time to hemostasis was relative to the final test/control article application. For test article treatments, additional test article application was required in 4 of 24 treatments (16.7%) to achieve hemostasis (i.e., a second piece of patch for these cases), mainly because the patch was not fully covering the bleeding site. For control article treatments, additional control article application was required in 3 of 8 treatments (37.5%) (i.e., a second piece of patch in 2 cases and a third piece in 1 case), mainly because of no adhesion of the patch to the tissue. No success criteria were established for endpoint 5. However, in summary, for test article treatments, the time to hemostasis was 30 seconds for 95.8% (23 of 24) treatments and was 5 minutes for 4.2% (1 of 24) treatments. For control article treatments, the time to hemostasis was 30 seconds for 100% (8 of 8) treatments. Based on these results, the time to hemostasis was comparable between test and control article treatments.

### Endpoint 6: Adhesion Formation

Adhesion formation was evaluated and given an adhesion extent/severity score. Observed adhesions were also collected and evaluated for remnants of the study article and/or other tissue response. No success criteria were established for endpoint 6, however in summary, at termination all test and control article treatment sites received an adhesion score of “1”, which was defined as thin, filmy adhesion which could be disrupted with minimal digital manipulation. This suggests that adhesions were negligible to the test article treatment sites and the response was no different from the control article treatment sites.

### Endpoint 7: Local Tissue Response to the Study Articles

Local tissue response to the study articles was assessed at gross necropsy by evaluating study article treated tissues for any clinically significant abnormalities. At gross necropsy assessment 1 of 24 (4.2%) Test Article treatment sites were evaluated as having remaining study article present, while 7 of 8 (87.5%) Control Article treatment sites were evaluated as having remaining study article present. Additionally, local tissue response to the study articles was evaluated using histopathology evaluations of inflammatory response, mineralization, and amount of study article remaining at the treatment site. With histopathology, remaining Test Article was observed in 8/24 sections, where all 8 sites were observed to have 1-25% Test Article remaining compared to the amount at baseline. Examining this further, 1 site had ˜1%, 5 sites had ˜1-5%, 1 site had ˜5%, and 1 site had ˜5-10% of Test Article remaining. Mineralization was observed in 3/24 sections, foreign debris in 16/24 sections, and hemorrhage in 22/24 sections. Remaining Control Article was observed in ⅜ sections and was morphologically deeply basophilic, where 2 sites were observed to have 1-25% and 1 site to have 76-100% of Control Article compared to the amount at baseline. Mineralization was observed in ⅛ sections, foreign debris in 6/8 sections, and hemorrhage in ⅞ sections. No success criteria were established for endpoint 7, however, in summary, all recorded gross lesions likely reflect regions of inflammation and fibrosis and are expected findings given the model. Mononuclear cell infiltrates are also common background findings in pigs and are not considered to be related to the Test Article. Similar amounts of inflammation (and reactivity scores), mineralization, foreign debris, and hemorrhage were observed between Control and Test Article sites. There were no significant or unexpected findings within the hepatic lymph nodes. Histologic evidence of remaining Test Article was observed in similar rates between the Test Article (33.3%) and Control Article (37.5%), however the amounts of Test Article remaining were lower than the amounts of Control Article remaining.

In both the open and laparoscopic GLP study, Hemostatic Patch 200 and Comparative Device 4+Thrombin resulted in hemostasis at all bleeding sites: Hemostatic Patch 200 achieved hemostasis in 96% at 30 seconds and in 100% at 60 seconds in the laparoscopic GLP study versus 98.8% at 60 seconds and 100% at 2 minutes in the open GLP study. In the laparoscopic GLP study, there was the need for additional patches placed in both the Hemostatic Patch 200 (17%) and Comparative Device 4+Thrombin (38%) groups; rates for the requirement of additional patches for initial hemostasis in the open GLP study was n=0 for Hemostatic Patch 200 and n=2 for Comparative Device 4+Thrombin. In the Hemostatic Patch 200 group, this was because the initial Hemostatic Patch 200 did not cover the entire bleeding site; this was not observed in the open GLP study. In the Comparative Device 4+Thrombin group, this was because the patch failed to adhere well enough to the tissue; this was also observed in the open GLP study in one bleeding site (n= 1/80, 1.3%).

Despite hemostasis being achieved with both therapies and no rescue therapies were required, there was a larger need for additional patch placement in the laparoscopic GLP study versus the open GLP study for both the Hemostatic Patch 200 and Comparative Device 4+Thrombin groups. This is likely caused by the small size of the patches in relation to the biopsy punch size (e.g., a 2.5 cm by 2.5 cm patch on an 8 mm biopsy punch results in an overlap of non-bleeding tissue of only 8.5 mm if placed perfectly, unlike the recommended 1 cm in the instructions for use of Hemostatic Patch 200); although the size of the patch was similar in the open GLP study, the open surgical approach allows for better visualization and more direct placement of the patches. Moreover, application in the laparoscopic setting is generally believed to be more difficult because the view and angle on the bleeding site is not always optimal. Moreover, the surgeon performing the surgery does not have current routine experience with laparoscopic surgery and some degree of imprecise application can therefore be expected. Indeed, an additional minimally invasive studies on Hemostatic Patch 200 found that in the acute laparoscopic non-GLP study performed by a surgeon that routinely performs laparoscopic surgery, a same-sized 2.5 by 2.5 cm Hemostatic Patch 200 was placed appropriately overlapping the bleeding site in all 16 of 16 same-sized 8 mm biopsy punch bleedings. Of note, in an additional study on use of Hemostatic Patch 200 in a robotic partial liver resection model, where the surgeon chose to apply larger parts of Hemostatic Patch 200 (range of 3 by 5 cm to 10 by 5 cm) with a sufficient overlap on clinically representable bleedings, all applications appropriately covered the bleeding site.

The completion of a usability questionnaire by 7 surgeons that performed different minimally invasive procedures with Hemostatic Patch 200 in the present studies found that all surgeons answered “Strongly agree” or “Agree” on a 5-Point Likert scale on the following statements about Hemostatic Patch 200:
Hemostatic Patch 200 could be introduced through a trocar without damaging, fragmenting, or rolling up to the degree that it can no longer be used (n=6 “Strongly Agree” and n=1 “Agree”)
Hemostatic Patch 200 could be navigated to the bleeding site without sticking to other structures or becoming too wet, to the degree that it can no longer be used (n=6 “Strongly Agree” and n=1 “Agree”)
Hemostatic Patch 200 could be adequately positioned on the bleeding site with minimally invasive tools (n=7 “Strongly Agree”)
Sufficient pressure could be provided with minimally invasive tools to adhere Hemostatic Patch 200 to the tissue (n=7 “Strongly Agree”).

In the open GLP study, there was only one case of rebleeding after Hemostatic Patch 200 application (e.g. 1.3%) that occurred during the procedure. In the laparoscopic GLP study there were no rebleedings occurring because of a hemostatic failure of Hemostatic Patch 200. There was one unintentional injury to a treatment site with a laparoscopic instrument that required a new Hemostatic Patch 200 application 40 minutes after the initial treatment. Of note, the assessment of rebleeding in the laparoscopic GLP Study included a 30-minute period of normal intra-abdominal pressure to mimic the direct postoperative period during which the risk of rebleeding is highest, and no rebleeding occurred during this period. There was no evidence of rebleeding at the 72-hrs sacrifice in the open GLP study and the 72-hrs laparoscopic relook in the laparoscopic GLP study, nor was there evidence of rebleeding at later timepoints of 4 and 8 weeks in both studies. In summary, the laparoscopic GLP study confirmed that in a minimally invasive setting the use of Hemostatic Patch 200 resulted in persistent hemostasis in a similar degree as when Hemostatic Patch 200 was used during open surgery.

In both the open and laparoscopic GLP studies, there was no evidence of product migration at any of the timepoints during surgery, at the 72-hrs sacrifice and laparoscopic relook, and at the later 4- and 8-week timepoints. The data from the laparoscopic GLP study provides evidence on appropriate pressure being applied during the application of Hemostatic Patch 200 so that it adequately adheres to the tissue and does not migrate, similar to the results in the open GLP study.

In the open GLP study, 72-hrs sacrifice identified overall gross adhesions to be present in 81% of Hemostatic Patch 200 sites and 86% of Comparative Device 4+Thrombin sites, as compared with 50% of Hemostatic Patch 200 sites and 100% of Comparative Device 4+Thrombin sites in the laparoscopic GLP study. In the open GLP study, these adhesions were all graded as ‘1’ (thin, filmy adhesion; disrupted with minimal digital manipulation). Because of the laparoscopic approach of the 3-day relook in the laparoscopic GLP study, no scoring of the nature of the adhesions was performed at this time. At the 4-week endpoint, the open GLP study found that gross adhesions were present in all Hemostatic Patch 200 and Comparative Device 4+Thrombin sites, and this was also found in the laparoscopic GLP study. The nature of the adhesions suggests that the laparoscopic GLP study was associated with a lower adhesions grading at the 4-week endpoint than the open GLP study. Of note, the open GLP study found at 8 weeks sacrifice that 83.3% of Hemostatic Patch 200 and 91% of Comparative Device 4+Thrombin sites had adhesion scores of ‘2’ and 8.3% and 9.0%, respectively, with a score of ‘3’, demonstrating that long-term adhesions are not expected to be more with Hemostatic Patch 200 versus Comparative Device 4+Thrombin.

With the majority of animals showing adhesions in the open and laparoscopic GLP study, irrespective of which hemostatic treatment is used, and overall clinical evidence on the existence of adhesions after intra-abdominal surgery for different indications, it can be summarized that Hemostatic Patch 200 by itself does not appear to be associated with an increase of adhesions post-surgery. Visual (gross) inspection of treatment sites at 4 weeks in the open and laparoscopic GLP study showed that the majority of treatment sites did not show any remnants of Hemostatic Patch 200, with rates of possible remnants being visible of 21% in the open GLP study and 4% in the laparoscopic GLP study.

During histopathology, it was confirmed in the open GLP study that degradation of Hemostatic Patch 200 occurred before the 4-week sacrifice of animals as there were no microscopical signs of Hemostatic Patch 200 on the bleeding sites. Although histological data is currently not yet available from the laparoscopic GLP study, the data on the visual inspection of the treatment sites suggest that the anticipated degradation time of 4 weeks is confirmed also in the laparoscopic GLP study.

The results from the laparoscopic GLP study confirm that Hemostatic Patch 200 can be satisfactorily introduced through a minimally invasive trocar, navigated to the bleeding site, and applied to the bleeding; the surgeon rates all data elements regarding usability as ‘acceptable’. As compared to Hemostatic Patch 200 use in an open surgical setting, laparoscopic use of Hemostatic Patch 200 demonstrated similar performance to the open GLP study in terms of hemostasis, rebleeding, product migration, adhesion formation, and product degradation. Based on these initial results, minimally invasive use of Hemostatic Patch 200 is considered safe and effective, but additional clinical and histopathological results in the final report of the laparoscopic GLP study may be expected to substantiate this conclusion further.

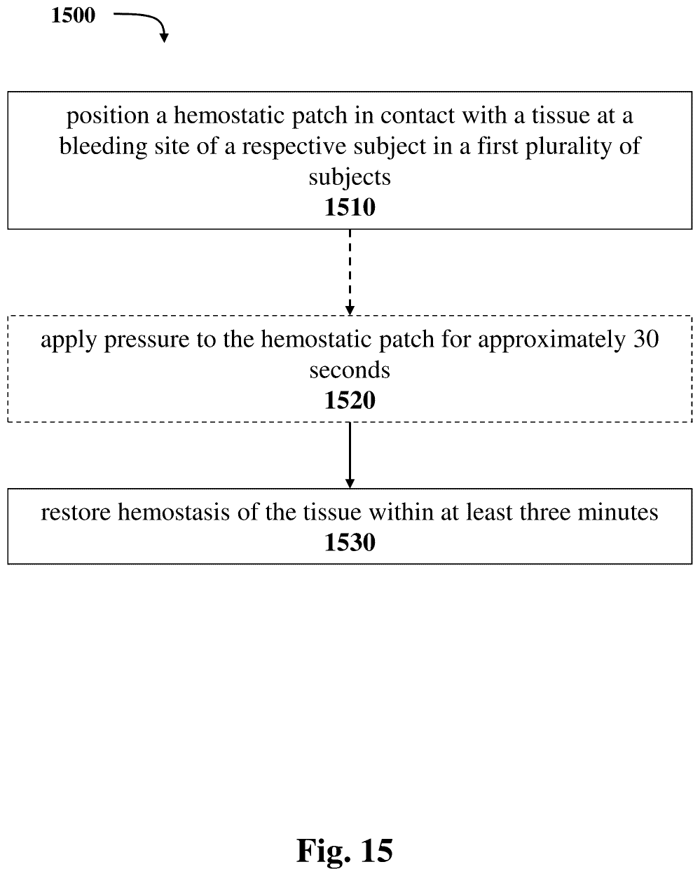

FIG. 15 depicts a method 1500 for treating hemorrhage in a subject during an open surgical procedure. The method 1500 can include delivering a Hemostatic Patch 200 in contact with a tissue at a bleeding site of a respective subject in a first plurality of subjects at step 1510. Although not depicted in method 1500, the operator can further position Hemostatic Patch 200 within a cavity at the bleeding site. Method 1500 can optionally include applying pressure to Hemostatic Patch 200 for approximately 30 seconds at step 1520. Method 1500 further includes restoring hemostasis of the tissue within at least three minutes at step 1530.

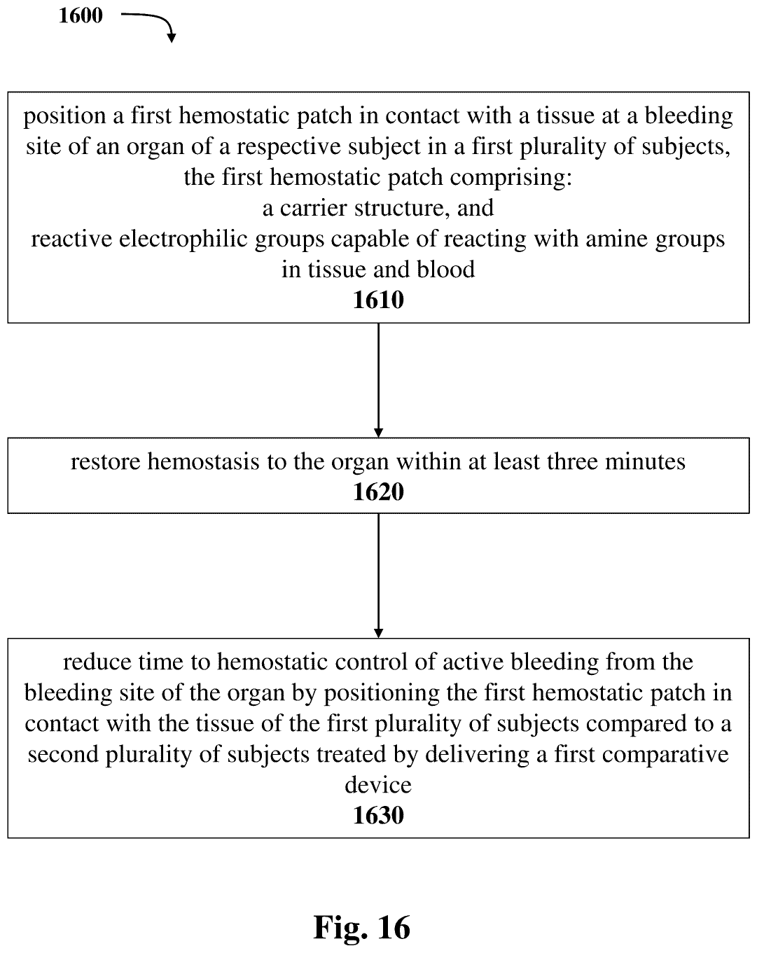

FIG. 16 depicts a method 1600 for treating hemorrhage in a subject during an open surgical procedure. The method 1600 can include positioning a first Hemostatic Patch 200 in contact with a tissue at a bleeding site of an organ of a respective subject in a first plurality of subjects at step 1610. The first Hemostatic Patch 200 can include a carrier structure, and reactive electrophilic groups capable of reacting with amine groups in tissue and blood. Although not depicted in method 1600, the operator can further position Hemostatic Patch 200 within a cavity at the bleeding site. Method 1600 can optionally include applying pressure to Hemostatic Patch 200 for approximately 30 seconds as described supra. Method 1600 further includes reducing time to hemostatic control of active bleeding from the bleeding site of the organ by positioning the first Hemostatic Patch 200 in contact with the tissue of the first plurality of subjects compared to a second plurality of subjects treated by delivering a first comparative device at step 1630. First comparative device can include Comparative Device 1 as described in more detail with respect to the second study described herein.

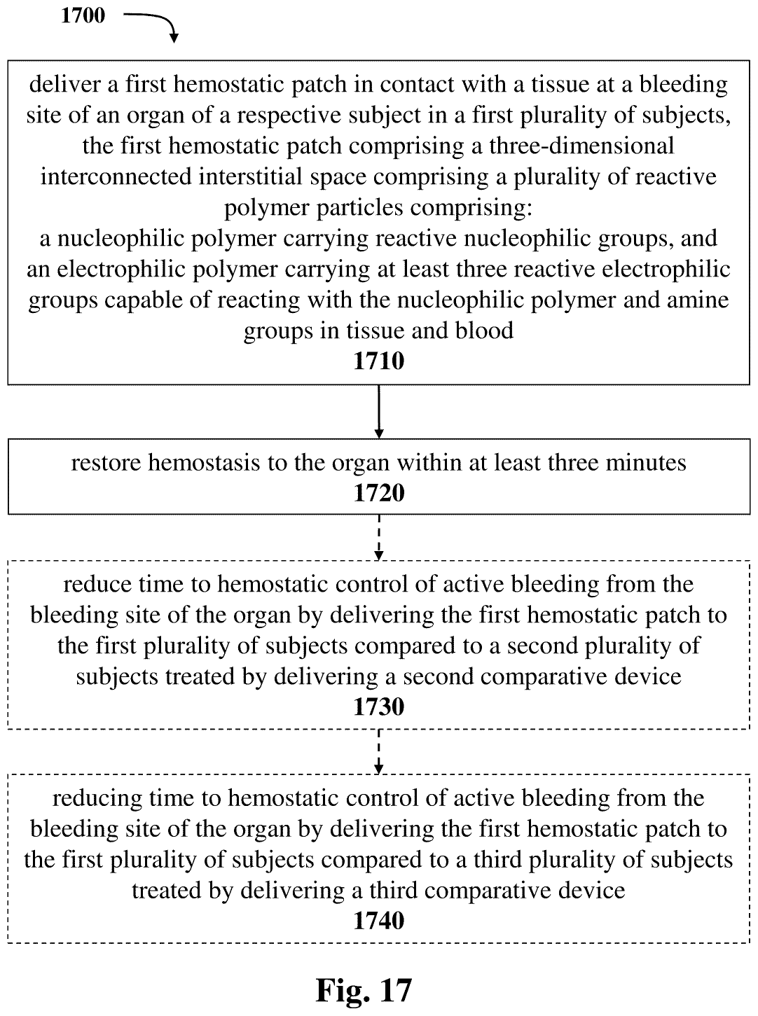

FIG. 17 depicts a method 1700 for treating hemorrhage in a subject during an open surgical procedure. The method 1700 can include delivering a first Hemostatic Patch 200 in contact with a tissue at a bleeding site of an organ of a respective subject in a first plurality of subjects at step 1710. The first Hemostatic Patch 200 includes a three-dimensional interconnected interstitial space having a plurality of reactive polymer particles. The reactive polymer particles, shown in more detail with reference to FIGS. 2A through 2C include a nucleophilic polymer carrying reactive nucleophilic groups, and an electrophilic polymer carrying at least three reactive electrophilic groups capable of reacting with the nucleophilic polymer and amine groups in tissue and blood. Although not depicted in method 1700, the operator can further position Hemostatic Patch 200 within a cavity at the bleeding site. Method 1700 can optionally include applying pressure to Hemostatic Patch 200 for approximately 30 seconds (step 1720) as described supra. Method 1700 further includes reducing time to hemostatic control of active bleeding from the bleeding site of the organ by positioning the first Hemostatic Patch 200 in contact with the tissue of the first plurality of subjects compared to a second plurality of subjects treated by delivering a second comparative device at step 1730. Alternatively, or in addition thereto, method 1700 includes reducing time to hemostatic control of active bleeding from the bleeding site of the organ by positioning the first Hemostatic Patch 200 in contact with the tissue of the first plurality of subjects compared to a third plurality of subjects treated by delivering a third comparative device at step 1740. Second comparative device can include Comparative Device 2 and third comparative device can include Comparative Device 3, as described in more detail with respect to the third study described herein.

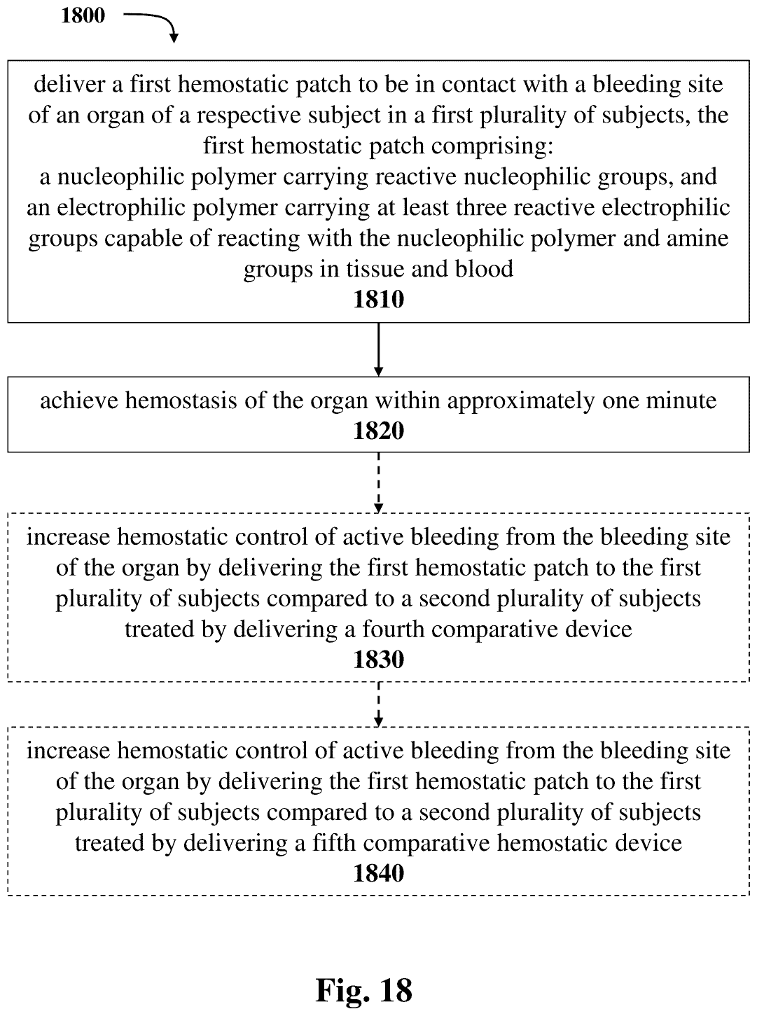

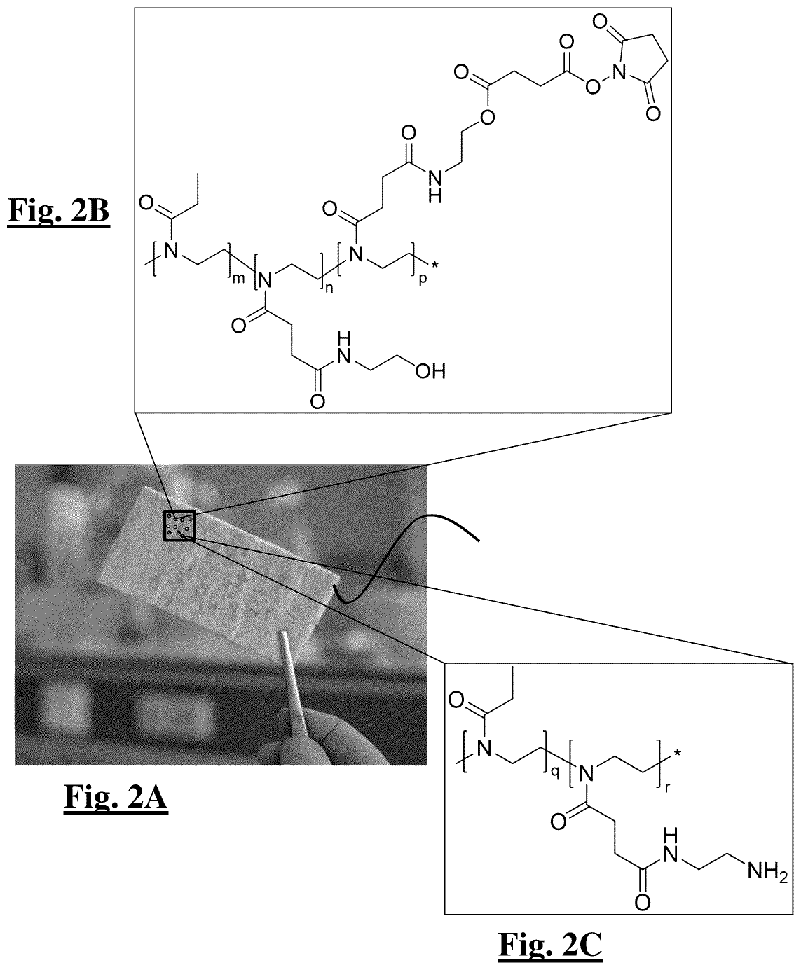

FIG. 18 depicts a method 1800 for treating hemorrhage in a subject during an open surgical procedure. The method 1800 can include delivering a first Hemostatic Patch 200 to be in contact with a bleeding site of an organ of a respective subject in a first plurality of subjects at step 1810. The first Hemostatic Patch 200 includes a three-dimensional interconnected interstitial space having a plurality of reactive polymer particles. The reactive polymer particles, shown in more detail with reference to FIGS. 2A through 2C include a nucleophilic polymer carrying reactive nucleophilic groups, and an electrophilic polymer carrying at least three reactive electrophilic groups capable of reacting with the nucleophilic polymer and amine groups in tissue and blood. Although not depicted in method 1800, the operator can further position Hemostatic Patch 200 within a cavity at the bleeding site. Method 1800 can optionally include applying pressure to Hemostatic Patch 200 for approximately 30 seconds (step 1820) as described supra. Method 1800 further includes reducing time to hemostatic control of active bleeding from the bleeding site of the organ by positioning the first Hemostatic Patch 200 in contact with the tissue of the first plurality of subjects compared to a second plurality of subjects treated by delivering a fourth comparative device at step 1830. Alternatively, or in addition thereto, method 1800 includes reducing time to hemostatic control of active bleeding from the bleeding site of the organ by positioning the first Hemostatic Patch 200 in contact with the tissue of the first plurality of subjects compared to a third plurality of subjects treated by delivering a fifth comparative device at step 1840. Fourth comparative device can include Comparative Device 4 and fifth comparative device can include Comparative Device 5, as described in more detail with respect to the fourth study described herein.

The disclosed technology described herein can be further understood according to the following clauses:

Clause 1: A method for treating hemorrhage in a subject during a surgical procedure, the method comprising: positioning a hemostatic patch in contact with a tissue at a bleeding site of a respective subject in a first plurality of subjects, the hemostatic patch comprising: a carrier structure, and reactive electrophilic groups capable of reacting with amine groups in tissue and blood; and restoring hemostasis of the tissue within at least three minutes.

Clause 2: The method of Clause 1, the hemostatic patch further comprising: a three-dimensional interconnected interstitial space comprising a plurality of reactive polymer particles comprising: an electrophilic polymer carrying the reactive electrophilic groups, and a nucleophilic cross-linking agent that contains reactive nucleophilic groups that are capable of reacting with the reactive electrophilic groups of the electrophilic polymer under the formation of a covalent bond.

Clause 3: The method of Clause 1 or 2, further comprising achieving hemostasis within approximately three minutes in at least 84.6% of subjects after positioning the hemostatic patch in contact with the tissue at the bleeding site of a respective subject.

Clause 4: The method of Clause 1 or 2, further comprising achieving hemostasis within approximately one minute in at least 81.4% of subjects after positioning the hemostatic patch in contact with the tissue at the bleeding site of a respective subject.

Clause 5: The method of Clause 1 or 2, further comprising achieving hemostasis within approximately 30 seconds in at least 65.9% of subjects after positioning the hemostatic patch in contact with the tissue at the bleeding site of a respective subject.

Clause 6: The method of any of Clauses 1-5, wherein the bleeding site is located in one of the following locations: liver, pancreas, spleen, stomach, gastrointestinal tract, kidney, bladder, reproductive organs, lungs, mediastinum, breast, lymph nodes, thymus, muscle, fat, heart, blood vessel, iliac artery, carotid artery, vena cava, or brain.

Clause 7: The method of any of Clauses 1-6, further comprising restoring hemostasis to the tissue presenting a bleeding severity equal to or less than 5 at the bleeding site of the tissue determined by a surface bleeding severity scale (SBSS).

Clause 8: The method of Clause 1, the hemostatic patch configured to fully degrade within approximately six weeks.

Clause 9: The method of Clause 8, further comprising allowing degradation of the hemostatic patch after restoring hemostasis to the tissue.

Clause 10: The method of any of Clauses 2-9, the electrophilic polymer comprising at least three reactive electrophilic groups that are capable of reacting with the nucleophilic cross-linking agent and amine groups in the tissue and blood.

Clause 11: The method of Clause 10, wherein the electrophilic polymer is selected from polyoxazolines, polyethylene glycols, polyvinylpyrrolidones, polyurethanes and combinations thereof.

Clause 12: The method of Clause 11, wherein the electrophilic polymer is a polyoxazoline.

Clause 13: The method of Clause 10, wherein the reactive electrophilic groups are selected from the group consisting of carboxylic acid esters, sulfonate esters, phosphonate esters, pentafluorophenyl esters, p-nitrophenyl esters, p-nitrothiophenyl esters, acid halide groups, anhydrides, ketones, aldehydes, isocyanato, thioisocyanato, isocyano, epoxides, activated hydroxyl groups, olefins, glycidyl ethers, carboxyl, succinimidyl esters, sulfo succinimidyl esters, maleimido (maleimidyl), ethenesulfonyl, imido esters, aceto acetate, halo acetal, orthopyridyl disulfide, dihydroxy-phenyl derivatives, vinyl, acrylate, acrylamide, iodoacetamide and combinations thereof.

Clause 14: The method of any of Clauses 10-13, the hemostatic patch comprising a molar ratio of electrophilic polymer to nucleophilic polymer ranging from about 1.0:0.10 to about 1.0:0.40.

Clause 15: The method of any of Clauses 1-14, the hemostatic patch further comprising a blue colorant.

Clause 16: A method for treating hemorrhage in a subject during a surgical procedure, the method comprising: positioning a first hemostatic patch in contact with a tissue at a bleeding site of an organ of a respective subject in a first plurality of subjects, the first hemostatic patch comprising: a carrier structure, and reactive electrophilic groups capable of reacting with amine groups in tissue and blood; and restoring hemostasis of the organ within at least three minutes.

Clause 17: The method of Clause 16, further comprising: reducing time to hemostatic control of active bleeding from the bleeding site of the organ by positioning the first hemostatic patch in contact with the tissue of the first plurality of subjects compared to a second plurality of subjects treated by delivering a first comparative device.

Clause 18: The method of Clause 17, further comprising achieving approximately 100% hemostasis within 8 minutes by delivering the first hemostatic patch to the first plurality of subjects.

Clause 19: The method of Clause 17, further comprising achieving approximately 88% hemostasis within 3 minutes or less by delivering the first hemostatic patch to the first plurality of subjects.

Clause 20: The method of Clause 19, further comprising achieving approximately 88% hemostasis within 30 seconds by delivering the first hemostatic patch to the first plurality of subjects.

Clause 21: The method of Clause 17, further comprising increasing degree of hemostasis control within 3 minutes or less by positioning the first hemostatic patch in contact with the tissue of the first plurality of subjects compared to the second plurality of subjects treated by delivering a first comparative device.

Clause 22: The method of any of Clauses 16-21, wherein the bleeding site is located in one of the following locations: liver, pancreas, spleen, stomach, gastrointestinal tract, kidney, bladder, reproductive organs, lungs, mediastinum, breast, lymph nodes, thymus, muscle, fat, heart, blood vessel, iliac artery, carotid artery, vena cava, or brain.

Clause 23: A method for treating hemorrhage in a subject during a surgical procedure, the method comprising: delivering a first hemostatic patch in contact with a tissue at a bleeding site of an organ of a respective subject in a first plurality of subjects, the first hemostatic patch comprising a three-dimensional interconnected interstitial space comprising a plurality of reactive polymer particles comprising: a nucleophilic polymer carrying reactive nucleophilic groups, and an electrophilic polymer carrying at least three reactive electrophilic groups capable of reacting with the nucleophilic polymer and amine groups in tissue and blood; and restoring hemostasis of the organ within at least three minutes.

Clause 24: The method of Clause 23, further comprising achieving hemostasis within about 10 seconds.

Clause 25: The method of Clause 23, further comprising reducing time to hemostatic control of active bleeding from the bleeding site of the organ by delivering the first hemostatic patch to the first plurality of subjects compared to a second plurality of subjects treated by delivering a second comparative device.

Clause 26: The method of Clause 25, further comprising achieving, by delivering the first hemostatic patch to the first plurality of subjects, increased hemostatic efficacy of active bleeding from a bleeding site of an organ compared to the second plurality of subjects treated by delivering the second comparative device.

Clause 27: The method of Clause 23, further comprising reducing time to hemostatic control of active bleeding from the bleeding site of the organ by delivering the first hemostatic patch to the first plurality of subjects compared to a third plurality of subjects treated by delivering a third comparative device.

Clause 28: The method of Clause 27, further comprising achieving, by delivering the first hemostatic patch to the first plurality of subjects, increased hemostatic efficacy of active bleeding from a bleeding site of an organ compared to the third plurality of subjects treated by delivering the third comparative device.

Clause 29: The method of any of Clauses 23-28, further comprising: positioning the first hemostatic patch in contact with a tissue at the bleeding site of the organ; and applying a pressure to the hemostatic patch while in contact with the tissue at the bleeding site of the organ.

Clause 30: The method of Clause 29, further comprising: achieving approximately 100% hemostasis within approximately 30 seconds by delivering the first hemostatic patch to the first plurality of subjects.

Clause 31: A method for treating hemorrhage in a subject during a surgical procedure, the method comprising: delivering a first hemostatic patch near or about a bleeding site of an organ of a respective subject in a first plurality of subjects, the first hemostatic patch comprising: a nucleophilic polymer carrying reactive nucleophilic groups, and an electrophilic polymer carrying at least three reactive electrophilic groups capable of reacting with the nucleophilic polymer and amine groups in tissue and blood; and achieving hemostasis of the organ within approximately one minute.

Clause 32: The method of Clause 31, further comprising achieving hemostasis within one minute for at least 94% of active bleeding sites.

Clause 33: The method of Clause 32, further comprising increasing hemostatic control of active bleeding from the bleeding site of the organ by delivering the first hemostatic patch to the first plurality of subjects compared to a second plurality of subjects treated by delivering a fourth comparative device.

Clause 34: The method of Clause 32, further comprising increasing hemostatic control of active bleeding from the bleeding site of the organ by delivering the first hemostatic patch to the first plurality of subjects compared to a second plurality of subjects treated by delivering a fifth comparative hemostatic device.

Clause 35: The method of any of Clauses 31-34, further comprising: positioning the first hemostatic patch in contact with a tissue at the bleeding site of the organ; and applying a pressure to the hemostatic patch for approximately 30 seconds while in contact with the tissue at the bleeding site of the organ.

Clause 36: The method of Clause 35, wherein the first hemostatic device is further configured to adhere to the organ until at least a portion of the first hemostatic patch biodegrades within approximately 6 weeks.

Clause 37: A device for treating surgical hemorrhage, the device comprising a biocompatible, flexible, hemostatic patch comprising: a nucleophilic polymer carrying reactive nucleophilic groups, and an electrophilic polymer carrying at least three reactive electrophilic groups capable of reacting with the nucleophilic polymer and amine groups in tissue and blood, the hemostatic patch configured to be delivered to an organ of a subject and restore hemostasis to the organ within approximately three minutes or less after positioning the hemostatic patch in contact with tissue at a bleeding site of the organ.

Clause 38: The device of Clause 37, wherein the electrophilic polymer is selected from polyoxazolines, polyethylene glycols, polyvinylpyrrolidones, polyurethanes and combinations thereof.

Clause 39: The device of Clause 37 or 38, wherein the electrophilic polymer is a polyoxazoline.

Clause 40: The device of any of Clauses 37-39, wherein the reactive electrophilic groups are selected from the group consisting of carboxylic acid esters, sulfonate esters, phosphonate esters, pentafluorophenyl esters, p-nitrophenyl esters, p-nitrothiophenyl esters, acid halide groups, anhydrides, ketones, aldehydes, isocyanato, thioisocyanato, isocyano, epoxides, activated hydroxyl groups, olefins, glycidyl ethers, carboxyl, succinimidyl esters, sulfo succinimidyl esters, maleimido (maleimidyl), ethenesulfonyl, imido esters, aceto acetate, halo acetal, orthopyridyl disulfide, dihydroxy-phenyl derivatives, vinyl, acrylate, acrylamide, iodoacetamide and combinations thereof.

Clause 41: The device of any of Clauses 37-39, wherein the hemostatic device is further configured to achieve hemostasis within approximately three minutes in at least 65.5% of subjects after positioning the hemostatic device near or about the bleeding site of the organ in a respective subject.

Clause 42: The device of any of Clauses 37-41, wherein the hemostatic device is further configured to achieve hemostasis within approximately three minutes in at least 84.6% of subjects after positioning the hemostatic device near or about the bleeding site of the organ in a respective subject.

Clause 43: The device of any of Clauses 37-42, wherein the hemostatic device is further configured to achieve hemostasis within approximately one minute in at least 81.4% of subjects after positioning the hemostatic device near or about the bleeding site of the organ in a respective subject.

Clause 44: The device of any of Clauses 37-43, wherein the hemostatic device is further configured to achieve hemostasis within approximately 30 seconds in at least 65.9% of subjects after positioning the hemostatic device near or about the bleeding site of the organ in a respective subject.

Clause 45: The device of any of Clauses 37-44, wherein the bleeding site is located in one of the following locations: liver, pancreas, spleen, stomach, gastrointestinal tract, kidney, bladder, reproductive organs, lungs, mediastinum, breast, lymph nodes, thymus, muscle, fat, heart, blood vessel, iliac artery, carotid artery, vena cava, or brain.

Clause 46: The device of any of Clauses 37-45, wherein the hemostatic device is further configured to adhere to the organ until at least a portion of the hemostatic device biodegrades within approximately 6 weeks.

Clause 47: The device of any of Clauses 37-46, wherein the hemostatic device is further configured to degrade after restoring hemostasis to the organ.

Clause 48: A biocompatible, flexible, hemostatic device for treating surgical hemorrhage, the hemostatic device comprising: a water-resistant cohesive fibrous carrier structure comprising: a three-dimensional interconnected interstitial space comprising a plurality of reactive polymer particles comprising an electrophilic polymer, and fibers comprising a nucleophilic polymer carrying reactive nucleophilic groups; and wherein the hemostatic device is capable of being delivered to an organ of a subject and restoring hemostasis to the organ within approximately three minutes or less by positioning the hemostatic device near or about a bleeding site of the organ.

Clause 49: The device of Clause 48, wherein the hemostatic device is further comprising a blue colorant.

Clause 50: The device of Clause 48 or 49, wherein the hemostatic device is further configured to achieve hemostasis within approximately three minutes in at least 84.6% of subjects after positioning the hemostatic device near or about the bleeding site of the organ in a respective subject.

Clause 51: The device of Clause 48, wherein the hemostatic device is further configured to achieve hemostasis within approximately one minute in at least 81.4% of subjects after positioning the hemostatic device near or about the bleeding site of the organ in a respective subject.

Clause 52: The device of Clause 48, wherein the hemostatic device is further configured to achieve hemostasis within approximately 30 seconds in at least 65.9% of subjects after positioning the hemostatic device near or about the bleeding site of the organ in a respective subject.

Clause 53: The device of Clause 48, wherein the bleeding site is located in one of the following locations: liver, pancreas, spleen, stomach, gastrointestinal tract, kidney, bladder, reproductive organs, lungs, mediastinum, breast, lymph nodes, thymus, muscle, fat, heart, blood vessel, iliac artery, carotid artery, vena cava, or brain.

Clause 54: The device of any of Clauses 48-53, wherein the hemostatic device is configured to restore hemostasis to the organ by adhering the hemostatic device in contact with a tissue at the bleeding site of the organ to a subject presenting a bleeding severity equal to or less than 3 at the bleeding site of the organ determined by a surface bleeding severity scale (SBSS).

Clause 55: The device of Clause 48, the electrophilic polymer comprises at least three reactive electrophilic groups that are capable of reacting with the nucleophilic polymer and amine groups in tissue of the organ and blood.

Clause 56: The device of Clause 55, the reactive polymer particles comprising a diameter in a range of about 0.5 μm to about 100 μm and being present in an amount of at least 3% by weight of the fibrous carrier structure.

Clause 57: The device of Clause 48, wherein the fibrous carrier structure is a felt structure, a woven structure, or a knitted structure.

Clause 58: The device of Clause 48, wherein the electrophilic polymer is selected from polyoxazolines, polyethylene glycols, polyvinylpyrrolidones, polyurethanes and combinations thereof.

Clause 59: The device of Clause 58, wherein the electrophilic polymer is a polyoxazoline.

Clause 60: The device of Clause 55, wherein the reactive electrophilic groups are selected from the group consisting of carboxylic acid esters, sulfonate esters, phosphonate esters, pentafluorophenyl esters, p-nitrophenyl esters, p-nitrothiophenyl esters, acid halide groups, anhydrides, ketones, aldehydes, isocyanato, thioisocyanato, isocyano, epoxides, activated hydroxyl groups, olefins, glycidyl ethers, carboxyl, succinimidyl esters, sulfo succinimidyl esters, maleimido (maleimidyl), ethenesulfonyl, imido esters, aceto acetate, halo acetal, orthopyridyl disulfide, dihydroxy-phenyl derivatives, vinyl, acrylate, acrylamide, iodoacetamide and combinations thereof.

Clause 61: The device of Clause 48, the hemostatic device comprising a molar ratio of electrophilic polymer to nucleophilic polymer ranging from about 1.0:0.10 to about 1.0:0.40.

The hemostatic device 200 and related methods of use of this disclosure demonstrated high rates of substantial hemostasis in patients with hemorrhage during minimally invasive procedures. The specific configurations, choice of materials and the size and shape of various elements can be varied according to particular design specifications or constraints requiring a system or method constructed according to the principles of the disclosed technology. Such changes are intended to be embraced within the scope of the disclosed technology. The presently disclosed embodiments, therefore, are considered in all respects to be illustrative and not restrictive. It will therefore be apparent from the foregoing that while particular forms of the disclosure have been illustrated and described, various modifications can be made without departing from the spirit and scope of the disclosure and all changes that come within the meaning and range of equivalents thereof are intended to be embraced therein.

What is claimed is:

1. A method for treating hemorrhage in a subject during a surgical procedure, the method comprising:
positioning a hemostatic patch in contact with a tissue at a bleeding site of a respective subject in a first plurality of subjects, the hemostatic patch comprising:
a carrier structure, and
reactive electrophilic groups capable of reacting with amine groups in tissue and blood; and
restoring hemostasis of the tissue within at least three minutes.

2. The method of claim 1, the hemostatic patch further comprising:
a three-dimensional interconnected interstitial space comprising a plurality of reactive polymer particles comprising:
an electrophilic polymer carrying the reactive electrophilic groups, and
a nucleophilic cross-linking agent that contains reactive nucleophilic groups that are capable of reacting with the reactive electrophilic groups of the electrophilic polymer under the formation of a covalent bond.

3. The method of claim 1, further comprising achieving hemostasis within approximately three minutes in at least 84.6% of subjects after positioning the hemostatic patch in contact with the tissue at the bleeding site of a respective subject.

4. The method of claim 1, further comprising achieving hemostasis within approximately one minute in at least 81.4% of subjects after positioning the hemostatic patch in contact with the tissue at the bleeding site of a respective subject.

5. The method of claim 1, further comprising achieving hemostasis within approximately 30 seconds in at least 65.9% of subjects after positioning the hemostatic patch in contact with the tissue at the bleeding site of a respective subject.

6. The method of claim 1, wherein:
the bleeding site is located in one of the following locations: liver, pancreas, spleen, stomach, gastrointestinal tract, kidney, bladder, reproductive organs, lungs, mediastinum, breast, lymph nodes, thymus, muscle, fat, heart, blood vessel, iliac artery, carotid artery, vena cava, or brain; and at least one of:
the hemostatic patch is configured to fully degrade within approximately six weeks;
the hemostatic patch is configured to degrade after restoring hemostasis to the tissue; and
the hemostatic patch further comprises a blue colorant.

7. The method of claim 1, further comprising restoring hemostasis to the tissue presenting a bleeding severity equal to or less than 5 at the bleeding site of the tissue determined by a surface bleeding severity scale (SBSS).

8. The method of claim 2, the electrophilic polymer comprising at least three reactive electrophilic groups that are capable of reacting with the nucleophilic cross-linking agent and amine groups in the tissue and blood.

9. The method of claim 8, wherein at least one of:
the electrophilic polymer is selected from polyoxazolines, polyethylene glycols, polyvinylpyrrolidones, polyurethanes and combinations thereof; and
the reactive electrophilic groups are selected from the group consisting of carboxylic acid esters, sulfonate esters, phosphonate esters, pentafluorophenyl esters, p-nitrophenyl esters, p-nitrothiophenyl esters, acid halide groups, anhydrides, ketones, aldehydes, isocyanato, thioisocyanato, isocyano, epoxides, activated hydroxyl groups, olefins, glycidyl ethers, carboxyl, succinimidyl esters, sulfo succinimidyl esters, maleimido (maleimidyl), ethenesulfonyl, imido esters, aceto acetate, halo acetal, orthopyridyl disulfide, dihydroxy-phenyl derivatives, vinyl, acrylate, acrylamide, iodoacetamide and combinations thereof.

10. The method of claim 9, wherein the electrophilic polymer is a polyoxazoline.

11. The method of claim 8, the hemostatic patch comprising a molar ratio of electrophilic polymer to nucleophilic polymer ranging from about 1.0:0.10 to about 1.0:0.40.

12. A method for treating hemorrhage in a subject during a surgical procedure, the method comprising:
positioning a first hemostatic patch in contact with a tissue at a bleeding site of an organ of a respective subject in a first plurality of subjects, the first hemostatic patch comprising:
a carrier structure, and
reactive electrophilic groups capable of reacting with amine groups in tissue and blood;
restoring hemostasis of the organ within at least three minutes, and
reducing time to hemostatic control of active bleeding from the bleeding site of the organ by positioning the first hemostatic patch in contact with the tissue of the first plurality of subjects compared to a second plurality of subjects treated by delivering a first comparative device.

13. The method of claim 12, further comprising
achieving approximately 100% hemostasis within 8 minutes by delivering the first hemostatic patch to the first plurality of subjects.

14. The method of claim 12, further comprising achieving approximately 88% hemostasis within 3 minutes or less by delivering the first hemostatic patch to the first plurality of subjects.

15. The method of claim 14, further comprising achieving approximately 88% hemostasis within 30 seconds by delivering the first hemostatic patch to the first plurality of subjects.

16. The method of claim 12, further comprising increasing degree of hemostasis control within 3 minutes or less by positioning the first hemostatic patch in contact with the tissue of the first plurality of subjects compared to the second plurality of subjects treated by delivering a first comparative device.

17. The method of claim 12, wherein the bleeding site is located in one of the following locations: liver, pancreas, spleen, stomach, gastrointestinal tract, kidney, bladder, reproductive organs, lungs, mediastinum, breast, lymph nodes, thymus, muscle, fat, heart, blood vessel, iliac artery, carotid artery, vena cava, or brain.

18. A device for treating surgical hemorrhage, the device comprising a biocompatible, flexible, hemostatic patch comprising:
a nucleophilic polymer carrying reactive nucleophilic groups, and
an electrophilic polymer carrying at least three reactive electrophilic groups capable of reacting with the nucleophilic polymer and amine groups in tissue and blood,
the hemostatic patch configured to be delivered to an organ of a subject and restore hemostasis to the organ within approximately three minutes or less after positioning the hemostatic patch in contact with tissue at a bleeding site of the organ.

19. The device of claim 18, wherein at least one of:
the electrophilic polymer is selected from polyoxazolines, polyethylene glycols, polyvinylpyrrolidones, polyurethanes and combinations thereof;
the reactive electrophilic groups are selected from the group consisting of carboxylic acid esters, sulfonate esters, phosphonate esters, pentafluorophenyl esters, p-nitrophenyl esters, p-nitrothiophenyl esters, acid halide groups, anhydrides, ketones, aldehydes, isocyanato, thioisocyanato, isocyano, epoxides, activated hydroxyl groups, olefins, glycidyl ethers, carboxyl, succinimidyl esters, sulfo succinimidyl esters, maleimido (maleimidyl), ethenesulfonyl, imido esters, aceto acetate, halo acetal, orthopyridyl disulfide, dihydroxy-phenyl derivatives, vinyl, acrylate, acrylamide, iodoacetamide and combinations thereof; and
the bleeding site is located in one of the following locations: liver, pancreas, spleen, stomach, gastrointestinal tract, kidney, bladder, reproductive organs, lungs, mediastinum, breast, lymph nodes, thymus, muscle, fat, heart, blood vessel, iliac artery, carotid artery, vena cava, or brain.

20. The device of claim 18, wherein the electrophilic polymer is a polyoxazoline.
